SEQUENCE LISTING (16 sequences)

SEQ ID NO: 1 - RNA, 23 nt, Artificial Sequence
  Synthetic Polynucleotide
aagauugacagacaccuaauauu

SEQ ID NO: 2 - DNA, 57 bp, Artificial Sequence
  Synthetic Polynucleotide
ccgggatgaccaagagatgctgtttctcgagaaacagcatctcttggtcatcttttt

SEQ ID NO: 3 - DNA, 57 bp, Artificial Sequence
  Synthetic Polynucleotide
ccggtgcaagatacaatggaggagactcgagtctcctccattgtatcttgcattttt

SEQ ID NO: 4 - DNA, 57 bp, Artificial Sequence
  Synthetic Polynucleotide
ccgggaagatgtgaagtcgtattatctcgagataatacgacttcacatcttcttttt

SEQ ID NO: 5 - DNA, 57 bp, Artificial Sequence
  Synthetic Polynucleotide
ccgggtgcagtagaatagacatcaactcgagttgatgtctattctactgcacttttt

SEQ ID NO: 6 - DNA, 57 bp, Artificial Sequence
  Synthetic Polynucleotide
ccggctcactttcattatactacctctcgagaggtagtataatgaaagtgagttttt

SEQ ID NO: 7 - DNA, 58 bp, Artificial Sequence
  Synthetic Polynucleotide
ccggattctcatacatggctataatctcgagattatagccatgtatgagaattttttg

SEQ ID NO: 8 - DNA, 58 bp, Artificial Sequence
  Synthetic Polynucleotide
ccggagaagatgtgaagtcgtattactcgagtaatacgacttcacatcttcttttttg

SEQ ID NO: 9 - DNA, 58 bp, Artificial Sequence
  Synthetic Polynucleotide
ccggatatgatcacagtcgtgttaactcgagttaacacgactgtgatcatattttttg

SEQ ID NO: 10 - DNA, 58 bp, Artificial Sequence
  Synthetic Polynucleotide
ccgggtggagaaagaatcggttaaactcgagtttaaccgattctttctccactttttg

SEQ ID NO: 11 - DNA, 58 bp, Artificial Sequence
  Synthetic Polynucleotide
ccggtatgatcacagtcgtgttaaactcgagtttaacacgactgtgatcatatttttg

SEQ ID NO: 12 - DNA, 57 bp, Artificial Sequence
  Synthetic Polynucleotide
ccgggccaagattgacagacacctactcgagtaggtgtctgtcaatcttggcttttt

SEQ ID NO: 13 - DNA, 58 bp, Artificial Sequence
  Synthetic Polynucleotide
ccgggtgcagtagaatagacatcaactcgagttgatgtctattctactgcactttttg

SEQ ID NO: 14 - RNA, 21 nt, Artificial Sequence
  Synthetic Polynucleotide
aagcccauaugaucacagucg

SEQ ID NO: 15 - protein, 8 aa, Artificial Sequence
  Synthetic Polypeptide
SIINFEKL

SEQ ID NO: 16 - protein, 8 aa, Artificial Sequence
  Synthetic Polypeptide
SIYNFEKL

(19) United States
(12) Patent Application Publication
(10) Pub. No.: US 2021/0052648 A1
(43) Pub. Date: Feb. 25, 2021

(54) METHOD OF PRODUCING LEUKOCYTES USING PTPN2 INHIBITION FOR ADOPTIVE CELL TRANSFER

(71) Applicant: MONASH UNIVERSITY, Clayton (AU)

(72) Inventors: Tony Tiganis, Clayton (AU); Florian Wiede, Clayton (AU)

(21) Appl. No.: 17/014,737

(22) Filed: Sep. 8, 2020

Related U.S. Application Data
(63) Continuation of application No. 15/317,197, filed on Dec. 8, 2016.

(30) Foreign Application Priority Data
Jun. 10, 2014 (AU) 2014902203
Mar. 31, 2015 (AU) 2015901171

(51) Int. Cl. A61K 35/17; C12N 15/113; C12N 5/0783; A61K 39/00; C12N 9/16; C12N 9/22

(52) U.S. Cl. CPC A61K 35/17; C12Y 301/03048; C12N 15/1137; C12N 5/0638; A61K 39/0011; C12N 9/16; A61K 2035/124; C12N 2310/14; C12N 2501/51; C12N 2501/515; C12N 2501/73; A61K 2039/5158; A61K 2039/572; C12N 9/22

(57) ABSTRACT

The present invention generally relates to methods of preparing leukocytes, particularly T cells, ex vivo for use in immunotherapy, particularly cancer immunotherapy. More specifically, the invention relates to methods for the preparation of leukocytes exhibiting cytotoxic properties for use in adoptive cell transfer. The invention also relates to cells and compositions including them for cancer immunotherapy. The invention also relates to methods of immunotherapy, particularly cancer immunotherapy. The present invention relates to a method for producing a leukocyte that has an enhanced capacity for killing a target cell, the method including contacting the leukocyte with a PTPN2 inhibitor in conditions for enabling the inhibitor to inactivate PTPN2 in the leukocyte, thereby producing a leukocyte that has an enhanced capacity for killing a target cell. Preferably, the leukocyte is contacted with the PTPN2 inhibitor in the absence of a T helper cell.

### CROSS REFERENCE

This application claims priority to Australian provisional application nos. 2014902203 and 2015901171, the entire contents of each are herein incorporated by reference.

### FIELD OF THE INVENTION

The present invention generally relates to methods of preparing cells ex vivo for use in immunotherapy, particularly cancer immunotherapy. More specifically, the invention relates to methods for the preparation of leukocytes, particularly T cells, exhibiting cytotoxic properties for use in adoptive cell transfer. The invention also relates to cells and compositions including them for cancer immunotherapy. The invention also relates to methods of immunotherapy, particularly cancer immunotherapy.

### BACKGROUND OF THE INVENTION

Immunotherapy is the use of the immune system of a patient to reject a disease, such as cancer or viral infection, by stimulating the patient's immune system to attack the malignant tumour or virally infected cells (and spare the normal cells of the patient). One mode of immunotherapy employs immunization of the patient (e.g., by administering a cancer vaccine) to train the patient's immune system to recognize and destroy tumour cells. Another approach uses the administration of therapeutic antibodies, thereby recruiting the patient's immune system to destroy tumour cells. Cell-based immunotherapy is another approach, which involves immune cells such as the Natural killer Cells (NK cells), Lymphokine Activated killer cell (LAK), Cytotoxic T Lymphocytes (CTLs), Dendritic Cells (DC), etc.

Many kinds of tumour cells or viral infected cells are tolerated by the patient's own immune system, as they are the patient's own cells (e.g., they are self) and are not effectively recognised by the patient's immune system allowing the tumour or viral infected cells to grow and divide without proper regulatory control. In addition, tumour-specific T cells are normally tolerized so that they do not respond to tumour activity. Accordingly, the patient's own immune system requires stimulation to attack the diseased cells.

Adoptive cell transfer (ACT) is an effective form of immunotherapy and involves the transfer of immune cells with anti-tumour or anti-viral activity into patients. ACT is a treatment approach that typically involves the identification of lymphocytes with anti-tumour or anti-viral activity, the in vitro expansion of these cells to large numbers and their infusion into the disease bearing host.

Adoptive T cell therapy depends on the ability to optimally select or genetically engineer cells with targeted antigen specificity and then induce the T cells to proliferate while preserving their effector function and engraftment and homing abilities. However, clinical trials have been carried out with adoptively transferred cells that were cultured in what are now understood to be suboptimal conditions that impair the essential functions of T cells such as antigen specific cytotoxic activity.

The methods which are currently used to prepare cells for use in adoptive cell therapy are limited in that they provide cells that have less than the expected cell killing of target cells, such as tumour cells.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention addresses one or more problems outlined above.

The present invention relates to a method for producing a leukocyte that has an enhanced capacity for killing a target cell, the method including
contacting the leukocyte with a PTPN2 inhibitor in conditions for enabling the inhibitor to inactivate PTPN2 in the leukocyte,
   thereby producing a leukocyte that has an enhanced capacity for killing a target cell. Preferably, the leukocyte is contacted with the PTPN2 inhibitor in the absence of a T helper cell.

The present invention relates to a method for producing a leukocyte cell that has an enhanced capacity for killing a target cell, the method including
contacting the leukocyte ex vivo with a PTPN2 inhibitor for a sufficient time and under conditions for inactivation of PTPN2 in the leukocyte,
   thereby producing a leukocyte cell that has an enhanced capacity for killing a target cell. Preferably, the leukocyte is not contacted ex vivo with a CD4 T helper cell.

The present invention relates to a method for preparing an ex vivo population of T cells exhibiting at least one property of a cytotoxic T cell including culturing T cells in the presence of a PTPN2 inhibitor.

The present invention relates to a method for preparing an ex vivo population of T cells exhibiting at least one property of a cytotoxic T cell including the steps of:
culturing a T cell population from a biological sample in the presence of a PTPN2 inhibitor;
expanding the cells in culture;

thereby preparing an ex vivo population of T cells exhibiting cytotoxic properties. Preferably the biological sample is derived from a subject having a cancer or have been conditioned or engineered to have specificity for a cancer.

The present invention relates to an ex vivo method for preparing a composition including antigen-specific cytotoxic T cells ex vivo including:
providing a biological sample containing T cells;
co-culturing antigenic material with the T cell population in the presence of a PTPN2 inhibitor; and
expanding the cells in culture,

thereby preparing a composition including antigen-specific cytotoxic T cells ex vivo.

The present invention relates to a method for increasing the level of T cells in a subject exhibiting an effector memory phenotype including the steps of:
culturing a T cell population from a biological sample ex vivo in the presence of a PTPN2 inhibitor;
expanding the cells in culture;
administering the cultured cells to the subject;
thereby increasing the level of T cells in a subject exhibiting an effector memory phenotype.

The present invention also provides a method for forming an immune response in a subject suitable for the treatment of cancer including the steps of
obtaining T cells from the subject or a histocompatible donor subject;
culturing the T cells in the presence of a PTPN2 inhibitor ex vivo for a sufficient time and under conditions for to generate a population of T cells exhibiting at least one cytotoxic T cell property, thereby forming a population of cytotoxic T cells,
administering the population of cytotoxic T cells to the subject,

thereby producing an immune response in a subject suitable for the treatment of cancer.

The present invention also relates to a method of increasing CD8+ T cell mediated immunity in a subject having a disease state including:
contacting CD8+ T cells with a PTPN2 inhibitor ex vivo for a sufficient time and under conditions to generate a population of CD8+ T cells exhibiting at least one property of a cytotoxic T cell;
administering the population of CD8+ T cells to the subject,

thereby increasing CD8+ T cell mediated immunity in a subject.

The present invention also relates to a method of increasing CD8+ T cell mediated immunity in a subject having a disease state including:
isolating a population of the subject's CD8+ T cells;
introducing a nucleic acid molecule encoding an siRNA or shRNA directed to PTPN2 into the isolated CD8+ T cells, thereby reducing the level of PTPN2 in a CD8+ T cell; and
reintroducing the CD8+ T cells into said subject,

thereby increasing the CD8+ T cell mediated immunity in a subject.

The present invention relates to a method of promoting regression of a cancer in a subject including the steps of:
culturing T cells obtained from a subject in the presence of a PTPN2 inhibitor,
administering the cultured T cells to the subject,

whereupon regression of the cancer is promoted.

The present invention relates to a method of promoting regression of a cancer in a subject having cancer including the steps of:
culturing CAR T cells specific for a tumour antigen expressed by the cancer in the presence of a PTPN2 inhibitor,
administering the cultured CAR T cells to the subject,

whereupon regression of the cancer is promoted. Preferably, the cancer is a Her-2 positive cancer and the CAR T cell is specific for Her-2.

The present invention relates to a method of prolonging survival of a subject having cancer including the steps of:
culturing CAR T cells specific for a tumour antigen expressed by the cancer in the presence of a PTPN2 inhibitor,
administering the cultured CAR T cells to the subject,

whereupon survival of the subject is prolonged. Preferably, the cancer is a Her-2 positive cancer and the CAR T cell is specific for Her-2.

The T cells may be selected from the group consisting of tumour infiltrating lymphocytes, peripheral blood lymphocyte, genetically engineered to express anti-tumour T cell receptors or chimeric antigen receptors (CARs), γδ T cells, enriched with mixed lymphocyte tumour cell cultures (MLTCs) or cloned using autologous antigen presenting cells and tumour derived peptides. The lymphocytes may be isolated from a histocompatible donor, or from the cancer-bearing subject.

In any method of the invention, the leukocytes or T cells are purified or substantially purified prior to culture in the presence of a PTPN2 inhibitor. This step enriches the leukocytes or T cells by removing other cell types from the biological sample.

In one embodiment, the CAR T cells are Her-2 specific CAR CD8+ T cells. The T cells may be a population that includes more than one type of T cells, including any one or more types described herein. For example, the population of T cells may include naïve, activated and/or memory T cells.

As used herein, a PTPN2 inhibitor may be any molecule that inhibits the phosphatase activity of PTPN2. The inhibitor may be a direct inhibitor of the phosphatase active site, may act allosterically to inhibit phosphatase activity, inhibit interaction of PTPN2 with its substrate, or may reduce the level of PTPN2 by reducing the transcriptional activity of the PTPN2 gene, or reducing the amount of PTPN2 mRNA or protein present in the cell. Typically, the inhibitor is a small molecule, for example ethyl-3,4-dephospatin or compound 8 as described herein, peptide, peptidomimetic, inhibitory or interferring RNA, such as antisense RNA, siRNA, microRNA or shRNA. Preferably, the siRNA has the sequence as shown in SEQ ID NO: 1. Preferably, the shRNA has the sequence shown in any one of SEQ ID NO: 2 to 12, or a sequence with at least 50%, 60%, 70&, 80%, 90%, 91%, 92%, 93%, 94%, 95%, 96%, 97%, 98% or 99% identity to any one of SEQ ID NO: 2 to 13 provided the shRNA still retains the ability to reduce PTPN2 levels in a cell.

The present invention also relates to tumour antigen-specific cytotoxic T cells for use in adoptive immunotherapy including an exogenous nucleic acid coding an interfering RNA, for example a microRNA, shRNA or siRNA, molecule that can reduce the level of PTPN2 in a cell.

The present invention relates to an isolated, purified or recombinant cell including an antigen-specific T cell receptor and an exogenous nucleic acid encoding an interfering RNA, for example a microRNA, shRNA or siRNA, molecule that can reduce the level of PTPN2 in a cell. Preferably, the TCR is specific for a cancer antigen and the cell is a CD8+ T cell. The CD8+ T cell may be a tumour infiltrating lymphocyte or a peripheral blood lymphocyte isolated from a host afflicted with cancer.

The present invention relates to a method of treating cancer in a subject including administering a population of isolated or purified CD8+ T cells effective to treat the cancer, the CD8+ T cell including an antigen-specific T cell receptor and an exogenous nucleic acid encoding an interfering RNA, for example a microRNA, shRNA or siRNA, molecule directed to PTPN2.

The present invention also provides a method for proliferating, enriching or expanding a composition of cells including a CD8+ T cell, the method including culturing a composition of cells in a medium, the medium including a PTPN2 inhibitor, wherein the PTPN2 inhibitor is provided in the medium to permit contact with a CD8+ T cell during culture. Preferably the proliferating, enriching or expanding will result in a doubling of the number of CD8+ T cells that exhibit at least one cytotoxic T cell property. More preferably the cell expansion result in 3× or 4× number of CD8+ T cells that exhibit at least one cytotoxic T cell property. The expansion of CD8+ T cells may be 5×, 6×, 7×, 8×, 9× or over 10×. The method may also increase the relative number of CD8+ T cells in the composition that exhibit at least one cytotoxic T cell property.

The present invention also relates to a composition of cytotoxic cells wherein greater than 20% of the cells have complete or partial inhibition of PTPN2. Preferably, the composition includes greater than 30%, 40%, 50%, 60%, 70%, 80%, 85%, 90%, 91%, 92%, 93%, 94%, 95%, 96%, 97%, 98 or 99% of cells that have complete or partial inhibition of PTPN2. In one embodiment, all cells have complete or partial inhibition of PTPN2.

The present invention also relates to a composition including a leukocyte and a PTPN2 inhibitor as described herein. Preferably, the PTPN2 inhibitor is an interfering RNA as described herein or ethyl-3,4-dephospatin or compound 8 as described herein. The composition may further include a cytokine for enhancing cell killing, such as IL-2 or IFNγ. Preferably, the leukocyte is a CAR T cell, more preferably the CAR T cell is specific for a cell surface tumour antigen. Typically, the CAR T cell is specific for Her-2.

In any aspect of the invention, the only inhibition is of PTPN2. In other words, no other gene or gene product other than PTPN2 is inhibited. For example, the only small molecule inhibitor used is a PTPN2 inhibitor or the only miRNA, shRNA or siRNA used targets PTPN2, or the only genome editing occurs to the PTPN2 gene.

As used herein, except where the context requires otherwise, the term “comprise” and variations of the term, such as “comprising”, “comprises” and “comprised”, are not intended to exclude further additives, components, integers or steps. The term “including” is also used interchangeably with “comprising” and is also not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. PTPN2-deficient naïve OT-I CD8+ T cells promote type I diabetes in RIPmOVA mice. a) Purified naïve (CD44^{lo}CD62L^{hi}) CD8⁺ lymph node (LN) T cells (8×10⁵) from OT-I:Ptpn2^{fl/fl} versus OT-I;Lck-Cre;Ptpn2^{fl/fl} mice were adoptively transferred into RIP-mOVA hosts and the survival and diabetes incidence (urine glucose ≥55 mmol/l) monitored. Blood glucose levels were assessed at days 7-10 post-transfer. b) Naive CD8⁺ LN T cells (4×10⁵) from OT-I:Ptpn2^{fl/fl} versus OT-I; Lck-Cre;Ptpn2^{fl/fl} mice were adoptively transferred into RIP-mOVA hosts and blood and urine glucose levels monitored at 15 days post transfer. Urine and blood glucose levels are means±SEM. Significant differences in urine and blood glucose levels were determined using 2-tailed Mann-Whitney U test (non-parametric); **P<0.01. Statistical analyses on Kaplan-Meier estimates in (a) (p<0.0001) and (b) (p=0.0012) were performed using a Log-rank (Mantel-Cox) test with one degree of freedom. c) Adoptive transfer of PTPN2-deficient naive OT-I CD8+ T cells into RIP-mOVA mice results in type 1 diabetes. Naive (CD44loCD62Lhi) CD8+ lymph node (LN) T cells (2.5×105) from OT-I:Ptpn2fl/fl versus OT-I; Lck-Cre; Ptpn2fl/fl mice were adoptively transferred into RIP-mOVA hosts and urine and blood glucose levels monitored after 15 days. Significant differences in urine and blood glucose levels were determined using 2-tailed Mann-Whitney U test (non-parametric); *p<0.05, **p<0.01.

FIG. 2. Adoptive transfer of PTPN2-deficient naive OT-I CD8+ T cells into RIPmOVA mice results in pancreatic β-cell destruction. Naive CD8⁺ LN T cells (8×10⁵) from OT-I:Ptpn2^{fl/fl} versus OT-I; Lck-Cre;Ptpn2^{fl/fl} mice were adoptively transferred into RIP-mOVA hosts. (a) At day 11 post-transfer pancreata were fixed in formalin and processed for histological assessment (hematoxylin and eosin: H&E). The severity of insulitis was determined histologically. The percent of islets graded 0-4 and those with invasive insulitis (grades 3 and 4) were determined for the indicated number of mice. Significant differences in invasive insulitis (means±SEM) were determined using 2-tailed Student's t-test; ***p<0.001. (b) Alternatively, fixed pancreata were processed for immunohistochemistry staining for insulin and glucagon.

FIG. 3. PTPN2-heterozygous naive OT-I CD8+ T cells cause pancreatic 3-cell destruction and diabetes in RIP-mOVA mice. Naive CD8+LN T cells (8×10⁵) from OT-I:Ptpn2^{fl/+} versus OT-I;Lck-Cre;Ptpn2^{fk/+} heterozygous mice were adoptively transferred into RIP-mOVA hosts. (a) Diabetes incidence was monitored (urine glucose ≥55 mmol/I). Statistical analyses on Kaplan-Meier estimates (p=0.0498) were performed using a Log-rank (Mantel-Cox) test with one degree of freedom. (b) At day 20 post-transfer pancreata were extracted, fixed in formalin and processed for histological assessment. The severity of insulitis was scored histologically. The percent of islets graded 0-4 and those with invasive insulitis (grades 3 and 4) were determined for the indicated number of mice. Significant differences in invasive insulitis (means±SEM) were determined using 2-tailed Student's t-test; *p<0.05.

FIG. 4. PTPN2-deficiency does not alter the proliferation of naive OT-I CD8+ T cells in RIP-mOVA. Naive CD8+LN T cells (2×10⁶) from OT-I:Ptpn2^{fl/fl} versus OT-I; Lck-Cre;Ptpn2^{fl/fl} mice were stained with FSE and adoptively transferred into RIP-mOVA hosts. At day 3 post-transfer lymphocytes were harvested from pancreatic, renal and inguinal LNs and stained for CD8 and TCR-Vα2 and analysed by flow cytometry. Representative FSE histograms of two independent experiments are shown. Quantified results are means±SEM for the indicated number of mice and are representative of two independent experiments.

FIG. 5. PTPN2-deficient naive OT-I CD8+ T cells show enhanced effector function after adoptive transfer into RIP-mOVA mice. Naive CD8+LN T cells (0.8×10⁶) from OT-I:Ptpn2^{fl/fl} versus OT-I; Lck-Cre;Ptpn2^{fl/fl} mice were adoptively transferred into RIP-mOVA hosts. At day 11 post-transfer lymphocytes were harvested from a) pancreatic and b) inguinal LNs and stained for CD8, TCR-Vα2 and TCR-Vβ5 and analysed by flow cytometry. Representative dot- and contour-plots of two independent experiments are shown. c) Naive CD8+LN T cells (2×10⁶) from OT-I:Ptpn2^{fl/fl} versus OT-I; Lck-Cre;Ptpn2^{fl/fl} mice were adoptively transferred into RIP-mOVA hosts. At day 9 post-transfer lymphocytes were harvested from pancreatic nodes and re-stimulated with PMA/lonomycin in the presence of Brefeldin A/Monensin, fixed and then permeabilized and stained with fluorochrome-conjugated antibodies against CD8, TCR-Vα2, TCR-Vβ5, IFNγ and granzyme B (Grz B) and analysed by flow cytometry. Representative contour-plots of two independent experiments are shown. Quantified results in (a-c) are means±SEM for the indicated number of mice and are representative of two independent experiments. In (a-c) significance was determined using 2-tailed Mann-Whitney U Test; *p<0.05, **p<0.01.

FIG. 6. PTPN2-deficiency enhances the activation of naive OT-I CD8+ T cells challenged with the cognate peptide SIINFEKL. a) CD8⁺ naive LN T cells from OT-I:Ptpn2^{fl/fl} versus OT-I; Lck-Cre;Ptpn2^{fl/fl} mice were stained with FSE and incubated with 0.1 μg/ml SIINFKL for 36 h and analysed by flow cytometry. Representative CFSE-histograms of two independent experiments are shown. b) CD8⁺ naive LN T cells from OT-I:Ptpn2^{fl/fl} versus OT-I; Lck-Cre;Ptpn2^{fl/fl} mice were incubated with 1 μg/ml SIINFKL for 18 h, stained for Annexin V and propidium-iodide (PI) and analysed by flow cytometry. c) CD8⁺ naive LN T cells from OT-I: Ptpn2^{fl/fl} versus OT-I;Lck-Cre;Ptpn2^{fl/fl} mice were incubated with 0.1 μg/ml SIINFKL for 36 h and stained for CD25, CD44, CD69 and CD62L and analysed by flow cytometry. Representative overlay-histograms from two independent experiments are shown. In (b-c) quantified results are means±SEM for the indicated number of mice and are representative of two independent experiments. In (c) significance was determined using 2-tailed Mann-Whitney U Test; *p<0.05, **p<0.01.

FIG. 7. PTPN2-deficiency enhances TCR- and IL-2-induced naive CD8+ T differentiation in vitro. a) CD8⁺ naive LN T cells from Ptpn2^{fl/fl} versus Lck-Cre; Ptpn2^{fl/fl} mice were stained with FSE and stimulated with plate-bound α-CD3 (10 μg/ml) and α-CD28 (5 μg/ml) for 48 h. Representative CFSE histograms of three independent experiments are shown. b) CD8⁺ naive LN T cells from Ptpn2^{fl/fl} versus Lck-Cre;Ptpn2^{fl/fl} mice were stimulated with α-CD3 (10 μg/ml) and α-CD28 (5 μg/ml) for 48 h and stained for CD25, CD44 and CD69 and analysed by flow cytometry. Representative overlay-histograms from two independent experiments are shown. c) Naive CD8⁺ LN T cells from Ptpn2^{fl/fl} versus Lck-Cre;Ptpn2^{fl/fl} mice were stimulated with plate-bound α-CD3 (10 μg/ml) and α-CD28 (5 μg/ml) for 48 h. At 2 days post-stimulation, cells were stained for CD44 and CD62L and processed for flow cytometry. Cells were incubated further with IL-2 (20 ng/ml) in the absence of α-CD3/α-CD28. At 2 and 4 days post IL-2 stimulation cells were stained for CD44 and CD62L and processed for flow cytometry to monitor the generation of CD44^{hi}CD62L^{lo} effector/memory T cells. Representative contour-plots of three independent experiments are shown. In (b) and (c) quantified results are means±SEM for the indicated number of mice and are representative of two independent experiments for (b) and three independent experiments for (c). In (b) and (c) significance was determined using 2-tailed Mann-Whitney U Test; **p<0.01, ***p<0.001.

FIG. 8. PTPN2-deficiency enhances the acquisition of an effector/memory phenotype in CD8+ CAR T cells ex vivo. Splenocytes from Lck-Cre;Ptpn2fl/fl (−/−) versus Ptpn2fl/fl (+/+) mice were stimulated and retrovirally co-transduced with scFv-anti-Her-2 and CD28-CD3-ζ (α-Her-2). After 8 days in culture Her-2 specific Lck-Cre;Ptpn2fl/fl (a-Her-2−/−) versus Ptpn2fl/fl control (α-Her-2+/+) CAR CD8+ T cells were stained with fluorochrome-conjugated antibodies against CD62L versus CD44 and the generation of effector/memory (CD44^{hi}CD62L^{lo}) was monitored by flow cytometry. On the left is Ptpn2^{fl/fl} (+/+) and the right Lck-Cre;Ptpn2^{fl/fl} (−/−).

FIG. 9. PTPN2-deficiency enhances CD8+ CAR T cell activation ex vivo. Her-2-specific Ptpn2^{fl/fl} (α-Her-2+/+) versus Lck-Cre;Ptpn2^{fl/fl} CD8⁺ CAR T cells (α-Her-2−/−) or non-transfected Ptpn2f (+/+) versus Lck-Cre;Ptpn2^{fl/fl} (−/−) CD8⁺ T cells were incubated with Her-2 expressing 24JK sarcoma cells (24JK-Her-2) or plate-bound a-CD3/CD28 (as a non-antigen-specific means by which to activate T cells). T cell activation was assessed by monitoring for secreted IFNg by ELISA.

FIG. 10. PTPN2-deficiency enhances CAR T cell CTL activity ex vivo. Her-2-specific Ptpn2^{fl/fl} (α-Her-2+/+) versus Lck-Cre;Ptpn2^{fl/fl} CAR CD8+ T cells (α-Her-2 −/−) CD8+ T cells were incubated with chromium (Cr⁵¹) labelled 24JK-Her-2 sarcoma cells or Her-2 negative 24JK sarcoma cells and Cr51 release measured four hours later.

FIG. 11. Inhibition of PTPN2-activity enhances polyclonal CD8+ T cell activation ex vivo. Purified CD8⁺ naive (CD62L^{hi}CD44^{lo}) splenic T cells isolated from C57BL/6 mice where incubated with plate-bound αCD3/CD28 for 48h in the presence or absence (vehicle) of a highly selective reversible PTPN2 inhibitor (compound 8). T cells were stained with fluorochrome-conjugated antibodies to assess CD44, CD62L, IL-2Rα (CD25 subunit) and CD69 surface levels and mean fluorescence intensity (MFI) was monitored by flow cytometry.

FIG. 12. PTPN2-deficiency enhances the conversion of naive T cells to effector/memory phenotype cells in vivo. Naive (CD62L^{hi}CD44^{lo}) CD8+LN T cells (2×10⁶) from CD45.1⁺ versus CD45.2⁺ Lck-Cre;Ptpn2fl/fl mice were co-transferred into replete CD45.1/2⁺ hosts. Peripheral blood was collected at the indicated time points post T cell transfer and the ratios of adoptively transferred CD8+ T cells from CD45.2 Lck-Cre;Ptpn2^{fl/fl} versus CD45.1 mice were determined by flow cytometry. At 16 weeks post-transfer recipient mice were sacrificed and lymphocytes from spleen, lymph node (LN), liver and lung were analyzed by flow cytometry. The ratios of adoptively transferred total and naive (CD62L^{hi}CD44^{lo}) central memory (CD62L^{hi}CD44^{hi}; CM) and effector/memory (CD62L^{lo}CD44^{hi}; EM) CD8+ T cells were determined. Results shown are means±SEM for the indicated number of mice.

FIG. 13. PTPN2-deficiency enhances the conversion of CD8+ central/memory to effector/memory T cells in vivo. Central memory (CD62L^{hi}CD44^{hi}, CM) CD8+LN T cells (0.5×10⁶) from CD45.1⁺ versus CD45.2⁺ Lck-Cre;Ptpn2^{fl/fl} mice were co-transferred into replete CD45.1/2+ hosts. Peripheral blood was collected at the indicated time points post T cell transfer and the ratios of adoptively transferred CD8+ T cells from CD45.2 Lck-Cre;Ptpn2^{fl/fl} versus CD45.1 mice were determined by flow cytometry. At 16 weeks post-transfer recipient mice were sacrificed and lymphocytes from spleen, lymph node (LN), liver and lung were analyzed by flow cytometry. The ratios of adoptively transferred total and naive (CD62L^{hi}CD44^{lo}) central memory (CD62L^{hi}CD44^{hi}; CM) and effector/memory (CD62L^{lo}CD44^{hi}; EM) CD8+ T cells were determined. Results shown are means±SEM for the indicated number of mice.

FIG. 14. Inhibition of PTPN2 in murine CD8+ T cells results in enhanced TCR-mediated T cell activation and conversion into effector/memory T cells. Naive (CD62LhiCD44lo) CD8+LN T cells (1×105) from C57BL/6 mice were stimulated with plate-bound anti-CD3 (5 μg/ml) and anti-CD28 (5 μg/ml) for 60h±PTPN2 inhibitor at various concentrations. Cells were harvested and stained with fluorochrome-conjugated antibodies for CD44, CD69, CD25 and CD62L and mean fluorescence intensity (MFI) analyzed by flow cytometry. Results shown are means±SD for the indicated number of replicates.

FIG. 15. PTPN2-deficiency does not enhance TCR-mediated activation-induced cell death in CD8+ T cells. CD8+ naive LN T cells (2×10⁵) from OT-I:Ptpn2^{fl/fl} versus OT-I; Lck-Cre;Ptpn2^{fl/fl} mice were incubated with 1 μg/ml SIINFEKL (N4) or 1 μg/ml SIYNFEKL (Y3) for 18 h, stained for Annexin V and propidium-iodide (PI) and analysed by flow cytometry. Results shown are means±SEM for the indicated number of mice.

FIG. 16. Inhibition of PTPN2 in CD8+ human blood lymphocytes results in enhanced TCR-mediated T cell activation. Freshly isolated human PBMCs (2×10⁶) were stimulated with plate-bound α-CD3 (1.25 and 2.5 μg/ml) for 24h. PBMCs were harvested and stained with fluorochrome-conjugated antibodies for CD45RA, CD8, CD69 and CD154 and T cell activation was monitored by flow cytometry. Representative CD8 versus CD69 and CD154 plots (numbers in outlined areas are the relative numbers in the gate) are shown.

FIG. 17. Knock down of PTPN2 using siRNAs in murine CD8+ T cells leads to enhanced TCR-mediated T cell responses. Splenocytes (1×10⁷) from C57BL/6 mice were transfected overnight with 100 nM GFP siRNA or 30 nM, 100 nM and 300 nM PTPN2 siRNA or 30 nM BLOCK-iT™ Fluorescent Oligo using the Amaxa Mouse T cell Nucleofactor Kit. (A) Transfection efficiency of CD8+ T cells was monitored with BLOCK-iT™ Fluorescent Oligo (Fluor.Oligo) by flow cytometry. (B) Transfected splenocytes were stimulated with plate-bound α-CD3 (5 μg/ml) and α-CD28 (5 μg/ml) for 48h and stained with fluorochrome-conjugated α-CD8. CD8+ T cell numbers were quantified with Calibrite Beads™ by flow cytometry. Results shown are means±SD for the indicated number of replicates.

FIG. 18. PTPN2-deficiency enhances the tumour-specific activity of Her-2 specific CAR T cells in the context of adoptive immunotherapy and prolongs the survival of xenografted mice. Her-2-specific Ptpn2^{fl/fl} (α-Her-2 Ptpn2^{+/+} CAR T cells) versus Lck-Cre; Ptpn2^{fl/fl} CAR T cells (α-Her-2 Ptpn2^{-/-} CAR T cells) (1×10⁷) were adoptively transferred into C57BL/6 human Her-2 transgenic mice (6 mice in each group) infected with Her-2 expressing 24JK-Her-2 sarcoma cells and survival was monitored. PTPN2-deficiency in Her-2 specific CAR T cells cured two out of six mice.

FIG. 19. Inhibition of PTPN2 in CD8+ human blood lymphocytes results in enhanced TCR-mediated T cell proliferation. Freshly isolated human PBMCs (2×10⁶) were stimulated with plate-bound α-CD3 for (A) 48h and (B) 72h, in the presence of vehicle control or the PTPN2 inhibitor, compound 8 (as described herein). PBMCs were harvested and stained with fluorochrome-conjugated α-CD8. Calibrite Beads™ were added and T cell proliferation was monitored by flow cytometry. Results shown are means±SD for the indicated number of replicates.

FIG. 20. Inhibition of PTPN2 enhances the tumour-specific activation of Her-2 specific CAR T cells ex vivo. Her-2-specific CAR T cells were incubated with Her-2 expressing 24JK sarcoma cells (24JK-Her-2) or 24JK sarcoma cells (24JK) or medium alone, in the presence of vehicle control or the PTPN2 inhibitor, compound 8 (described herein). T cell activation was assessed by monitoring for secreted IFNγ by ELISA (Mouse IFNγELISA Set, BD OptEIA™) according to the supplier's specifications. Tests were performed in triplicates (±SD).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The inventors have developed a method for the efficient preparation of cells for use in adoptive cell transfer, particularly for cancer immunotherapy. The inventors have surprisingly found that inhibiting the activity of PTPN2 in T cells enhances the capacity for killing a target cell. Further, an advantage of the present invention is that T cells which are tolerised but would otherwise be useful in adoptive cell transfer, for example as they are specific for tumour antigens in the case of tumour infiltrating lymphocytes, can be reinvigorated and tolerance reduced. A further advantage of a method of the present invention is that T cells can be differentiated down the cytotoxic CD8+ T cell lineage ex vivo without the need for the presence of CD4+ T cell help.

Without being bound by any theory or mode of action, it is believed that inhibition of PTPN2 activity causes alteration in T cell receptor (TCR) signalling thereby reversing or avoiding tolerance and instead promoting differentiation of T cells down the cytotoxic T cell lineage. For example, isolated CD8+ T cells treated so as to reduce PTPN2 activity lead to any one or more of the following functions: develop cytotoxic activity towards cells that bear an antigen to which an enhanced immune response would be desirable, enhanced sustenance and/or antigen-recall responses to presentation of the antigen, or have functional and/or phenotypic characteristics of effector T cells.

Although cancer immunotherapies of ex vivo cultured CD8+ T cells have been demonstrated to exhibit remarkable efficacy, such therapies are not effective in every patient as it is difficult to obtain an effective number of CD8+ T cells that have the ability to target the tumour cells and kill the tumour cell once recognised. The present invention provides a means for producing cells that have an enhanced capacity to kill a target cell, such as a tumour cell.

Antigen cross-presentation by dendritic cells is crucial for priming cytotoxic CD8+ T cells to invading pathogens and tumour antigens, as well as mediating peripheral tolerance to self-antigens. The protein tyrosine phosphatase N2 attenuates T cell receptor signaling and tunes CD8+ T cell responses in vivo. The inventors have examined the role of PTPN2 in the maintenance of peripheral tolerance after the cross-presentation of pancreatic β-cell antigens. The transfer of OVA-specific OTI CD8+ T cells (C57BL/6) into RIP-mOVA recipients expressing OVA in pancreatic β-cells only results in islet destruction when OVA-specific CD4+ T cells are co-transferred. The inventors show that PTPN2-deficient OT-I CD8+ T cells transferred into RIP-mOVA recipients acquire CTL activity and result in p cell destruction and the development of diabetes in the absence of CD4+ help. These studies identify PTPN2 as a critical mediator of peripheral T cell tolerance limiting CD8+ T cell responses after the cross-presentation of self-antigens. The findings reveal a mechanism by which PTPN2 deficiency might convert a tolerogenic CD8+ T cell response into one capable of causing the destruction of pancreatic β-cells. Moreover, the results provide insight into potential approaches for enhancing T cell-mediated immunity and/or T cell adoptive tumour immunotherapy.

The elimination or inactivation of T cells with auto-reactive potential is a critical task that is synergistically mediated by both thymic and peripheral tolerance mechanisms. The majority of auto-reactive T cells are eliminated in the thymus through negative selection; a process that is facilitated by the ability of the thymic medullary cells to ectopically express peripheral tissue antigens. Nonetheless, the few highly auto-reactive T cells that might escape this selection are subsequently eliminated by peripheral tolerance mechanisms.

A series of experiments have established that immature dendritic cells (DCs) play an essential role in this context. Immature DCs acquire self-antigens from non-inflamed tissues, transport them to lymph nodes and present them in a way that induces limited T cell expansion and finally the deletion of T cells that strongly react to the tissue-derived antigens. The induction of a cytotoxic T lymphocyte immune response to an invading pathogen begins in the LNs that drain the infection site and requires the processing and presentation of exogenous antigens by mature antigen-presenting cells (APCs). Notably, both the initiation of a CTL response and the tolerisation of auto-reactive T cells often depend on the capacity of DCs to acquire exogenous antigens and to channel peptide derived from these antigens onto their own MHC-1 molecules; a process referred to as cross-presentation.

CD4⁺ T cells have been shown to impact the cross-presenting and T cell stimulatory ability of DCs, as they are able to induce the maturation of DCs; a process known as T cell licensing. The latter is thought to be particularly critical in the absence of strong pro-inflammatory stimuli. Moreover, CD4 T cells have been shown to mature self-antigen presenting DCs and to thereby transform their tolerising potential into autoimmunity promoting cells.

Anatomic sources of leukocytes, preferably T cells, from a subject include peripheral blood, tumours, malignant effusions, and draining lymph nodes. Lymphocytes used for adoptive transfer can either be derived from the stroma of resected tumours (tumour infiltrating lymphocytes), or from blood and: genetically engineered to express antitumour T cell receptors or chimeric antigen receptors (CARs), enriched with mixed lymphocyte tumour cell cultures (MLTCs) or cloned using autologous antigen presenting cells and tumour derived peptides. The lymphocytes used for infusion can be isolated from an allogenic donor, preferably HLA matched, or from the cancer-bearing subject. In one embodiment, the leukocytes, preferably T cells, from a subject are not obtained or derived from the bone marrow.

In any method of the invention the leukocytes, preferably T cells, that have been cultured in the presence of a PTPN2 inhibitor can be transferred into the same mammal from which cells were obtained. In other words, the cells used in the a method of the invention can be an autologous cell, i.e., can be obtained from the mammal in which the medical condition is treated or prevented. Alternatively, the cell can be allogenically transferred into another subject. Preferably, the cell is autologous to the subject in a method of treating or preventing a medical condition in the subject.

One source of T cells targeted for cancer immunotherapy may be to use artificial chimeric receptors derived, for example, from the antigen binding domain of a monoclonal antibody. When coupled to appropriate intracellular signaling domains, T cells expressing these chimeric antigen receptors (CAR) can kill tumor cell targets. CAR T cells have the advantage of acting in a MHC unrestricted manner, allowing them to target tumor cells in which antigen processing or presentation pathways are disrupted. Moreover, they can be directed to nonpeptide antigens on the cell surface, broadening the range of target structures that can be recognized on malignant cells. Hence, CAR-expressing T cells can complement MHC restricted cytotoxic T cells, and increase the overall effectiveness of this cellular immunotherapy.

When naive CD8+ and CD4+ T cells engage peptide antigen presented by major histocompatibility complex (MHC) molecules, the T cell receptor signal strength determines whether T cells progress past the G₁ restriction point and commit to cellular division, produce interleukin-2 (IL-2) and undergo clonal expansion/proliferation and differentiate and acquire various effector functions. TCR signaling is reliant on tyrosine phosphorylation mediated by the Src family protein tyrosine kinases, Lck and Fyn, and the Syk family PTK ZAP-70. Engagement of the TCR allows for Lck to phosphorylate the immunoreceptor tyrosine-based activation motifs of the TCR that result ZAP-70 recruitment and activation and the phosphorylation of adaptor proteins such as LAT. This in turn allows for the nucleation of signaling complexes and the phosphorylation and activation of multiple effector pathways. Upon TCR engagement, the activation and/or functions of Lck are regulated by the localisation of Lck and its substrates, as well as the abundance, activity and segregation of regulatory molecules within the immunological synapse. Such regulatory molecules include protein tyrosine phosphatases (PTPs) that regulate the phosphorylation of the Lck Y505 inhibitory site, as well as the Lck Y394 activating site.

PTPN2 (also known as T cell PTP, PTN2, PTPT, TC-PTP, TCELLPTP and TCPTP) is a ubiquitous phosphatase that is expressed abundantly in hematopoietic cells, including T cells. Two splice variants of TCPTP are expressed that have identical N termini and catalytic domains but varied C termini: a 48-kDa form (TC48) that is targeted to the endoplasmic reticulum (ER) by a hydrophobic C terminus and a 45-kDa variant (TC45) that is targeted to the nucleus by a nuclear localization sequence. Despite an apparently exclusive nuclear localization in resting cells, TC45 can shuttle between the nucleus and cytoplasm to access substrates in both compartments. Genome-wide association studies have linked PTPN2 single nucleotide polymorphisms (SNPs) with the development of several human autoimmune diseases including type 1 diabetes, rheumatoid arthritis, Crohn's disease and celiac disease. In particular, an intronic PTPN2 variant, rs1893217(C), has been linked with the development of type 1 diabetes. This SNP is associated with an approximate 40% decrease in PTPN2 mRNA in CD4+ T cells. PTPN2 is a key regulator of TCR signaling in naive CD4+ and CD8+ T cells and functions to dephosphorylate and inactivate Lck and Fyn. PTPN2 also dephosphorylates Janus-activated kinases (JAK)-1/3 and signal transducers and activator of transcription (STAT)-1/3/5/6 to attenuate cytokine signaling.

In order to determine if the presence of a PTPN2 inhibitor has inhibited PTPN2, experiments such as the following could be performed: measure PTPN2 activity in PTPN2 immunoprecipitates using p-NPP (para-nitrophenylphosphate) and p-tyr-RCML (p-tyr-reduced, carboxyamidomethylated and maleylated lysozyme) as substrates as described previously (Bukczynska P et al. Biochem J. 2004 Jun. 15; 380(Pt 3):939-49; Tiganis T et al. J Biol Chem. 1997 Aug. 22; 272(34):21548-57). Alternatively, analysis of known substrates of PTPN2 such as Src-family kinase members Lck and Fyn and transcription factors STAT1, STAT3 and STAT5 for tyrosine-phosphorylation by flow cytometry and immuno-blotting can be performed.

A PTPN2 inhibitor useful in the present invention is one that completely or partially reduces one or more functions of PTPN2 as described herein. Preferably, a PTPN2 inhibitor reduces phosphatase activity of PTPN2 (such as a small molecule, peptide or peptidomimetic), reduces the transcriptional activity of the PTPN2 gene, or reduces the amount of PTPN2 mRNA or protein present in the cell. Exemplary small molecules that inhibit PTPN2 and that are useful in the present invention are ethyl-3,4-dephospatin or compound 8 (Zhang et al. (2009), JACS, 131, 13072 to 13079). Other inhibitors that may be useful in the invention include molecules with PTPN2 inhibitory activity as described in WO03/073987 A2; WO 03/097621 A1; US 2012/0088720 A1; U.S. Pat. No. 7,393,869; and US 2006/0235061 A1.

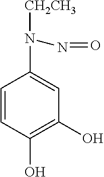 [SMILES: CCN(N=O)c1ccc(O)c(O)c1]

Chemical structure of ethyl-3,4-dephospatin.

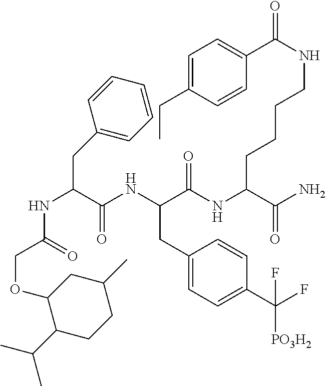

Chemical structure of compound 8.

The expression of PTPN2 can be reduced by any means that reduces the level of PTPN2 transcription. For example, miRNA, shRNA or siRNA approaches can be used. Exemplary siRNA and shRNA include any one or more of the following sequences or sequences having sufficient homology to reduce expression of PTPN2 by targeting the coding sequence of PTPN2 or the 3′UTR.

Exemplary siRNA includes:

SEQ ID NO: 1
(5═AAGAUUGACAGACACCUAAUAUU3═);
and
SEQ ID NO: 14
(5═AAGCCCAUAUGAUCACAGUCG3═);

and exemplary shRNA include:

TRCN0000002781, with a target sequence of GATGACCAAGAGATGCTGTTT beginning at position 582 of PTPN2 sequence from NM_001207013.1 and a hairpin sequence of:

SEQ ID NO: 2;
5′-CCGG-GATGACCAAGAGATGCTGTTT-CTCGAG-AAACAGCATCTCT
TGGTCATC-TTTTT-3′;

TRCN0000002782, with a target Sequence of TGCAAGATACAATGGAGGAGA beginning at position 1273 of PTPN2 sequence from NM_001207013.1 and a hairpin sequence of:

SEQ ID NO: 3;
5′-CCGG-TGCAAGATACAATGGAGGAGA-CTCGAG-TCTCCTCCATTGT
ATCTTGCA-TTTTT-3′;

TRCN0000002783, with a target sequence of GAAGATGTGAAGTCGTATTAT beginning at position 636 of PTPN2 sequence from NM_001207013.1 and a hairpin sequence of:

SEQ ID NO: 4;
5′-CCGG-GAAGATGTGAAGTCGTATTAT-CTCGAG-ATAATACGACTTC
ACATCTTC-TTTTT-3′;

TRCN0000002784, with a target sequence of GTGCAGTAGAATAGACATCAA beginning at position 1542 of PTPN2 sequence from NM_002828.3 and a hairpin sequence of:

SEQ ID NO: 5;
5′-CCGG-GTGCAGTAGAATAGACATCAA-CTCGAG-TTGATGTCTATTC
TACTGCAC-TTTTT-3′;

TRCN0000002785, with a target sequence of CTCACTTTCATTATACTACCT beginning at position 781 of PTPN2 sequence from NM_001207013.1 and a hairpin sequence of:

SEQ ID NO: 6;
5′-CCGG-CTCACTTTCATTATACTACCT-CTCGAG-AGGTAGTATAATG
AAAGTGAG-TTTTT-3′;

TRCN0000314692, with a target sequence of ATTCTCATACATGGCTATAAT beginning at position 1061 of PTPN2 sequence from NM_001207013.1 and a hairpin sequence of:

SEQ ID NO: 7;
5′-CCGG-ATTCTCATACATGGCTATAAT-CTCGAG-ATTATAGCCATGT
ATGAGAAT-TTTTTG-3′;

TRCN0000314609, with a target sequence of AGAAGATGTGAAGTCGTATTA beginning at position 635 of PTPN2 sequence from NM_001207013.1 and a hairpin sequence of:

SEQ ID NO: 8;
5′-CCGG-AGAAGATGTGAAGTCGTATTA-CTCGAG-TAATACGACTTCA
CATCTTCT-TTTTTG-3′;

TRCN0000279329, with a target sequence of ATATGATCACAGTCGTGTTAA beginning at position 270 of PTPN2 sequence from NM_001127177.1 and a hairpin sequence of:

SEQ ID NO: 9;
5′-CCGG-ATATGATCACAGTCGTGTTAA-CTCGAG-TTAACACGACTGT
GATCATAT-TTTTTG-3′;

TRCN0000314612, with a target sequence of GTGGAGAAAGAATCGGTTAAA beginning at position 540 of PTPN2 sequence from NM_001207013.1 and a hairpin sequence of:

SEQ ID NO: 10
5′-CCGG-GTGGAGAAAGAATCGGTTAAA-CTCGAG-TTTAACCGATTCT
TTCTCCAC-TTTTTG-3′;

TRCN0000314693, with a target sequence of TATGATCACAGTCGTGTTAAA beginning at position 354 of PTPN2 sequence from NM_001207013.1 and a hairpin sequence of:

SEQ ID NO: 11
5′-CCGG-TATGATCACAGTCGTGTTAAA-CTCGAG-TTTAACACGACTG
TGATCATA-TTTTTG-3′;

TRCN0000029891, with a target sequence of GCCAAGATTGACAGACACCTA beginning at position 8031 of PTPN2 sequence from NM_001127177.1 and a hairpin sequence of:

SEQ ID NO: 12
5′-CCGG-GCCAAGATTGACAGACACCTA-CTCGAG-TAGGTGTCTGTCA
ATCTTGGC-TTTTT-3′;

TRCN0000314551, with a target sequence of GTGCAGTAGAATAGACATCAA beginning at position 1542 of PTPN2 sequence from NM_002828.3 and a hairpin sequence of:

SEQ ID NO: 13.
S-CCGG-GTGCAGTAGAATAGACATCAA-CTCGAG-TTGATGTCTATTCT
ACTGCAC-TTTTTG-3′:

Further, the inhibition of PTPN2 may also include genome editing to remove or modify all or part of a sequence encoding PTPN2. An exemplary genome editing technique is the CRISPR/Cas9 system (Jinek, M., et al. (2012) Science, 337, 816-821; Cong L., et al. (2013) Science, 339, 819-823; and Qi, L. S., et al. (2013) Cell, 152, 1173-1183).

The miRNA, siRNA or shRNA can be delivered to the relevant T cell by using a viral vector. There are a large number of available viral vectors that are suitable for use with the present invention, including those identified for human gene therapy applications. Suitable viral vectors include vectors based on RNA viruses, such as retrovirus-derived vectors, e.g., Moloney murine leukemia virus (MLV)-derived vectors, and include more complex retrovirus-derived vectors, e.g., Lentivirus-derived vectors. Human Immunodeficiency virus (HIN-I)-derived vectors belong to this category. Other examples include lentivirus vectors derived from HIN-2, feline immunodeficiency virus (FIN), equine infectious anemia virus, simian immunodeficiency virus (SIV) and Maedi-Visna virus.

Preferably a modified retrovirus, even more preferably a modified lentivirus, is used to deliver the specific miRNA, siRNA or shRNA. This virus may also include sequences that encode the chimeric antigen T cell receptor for targeting the specific cell to be killed. The polynucleotide and any associated genetic elements are thus integrated into the genome of the host cell as a provirus. The modified retrovirus is preferably produced in a packaging cell from a viral vector that includes the sequences necessary for production of the virus as well as the miRNA, siRNA or shRNA and/or CAR. The viral vector may also include genetic elements that facilitate expression of the miRNA, siRNA or shRNA, such as promoter and enhancer sequences. In order to prevent replication in the target cell, endogenous viral genes required for replication may be removed.

A composition including the CD8+ T cells and the PTPN2 inhibitor may further include the cancer specific antigen and/or one or more cytokines to enhance cell killing (such as IL-2 or IFNγ). When the antigen is present in the composition including the isolated, enriched or purified CD8+ T cells, the antigen may be present as an independent entity, or in any context by which the antigen can interact with the T cell receptor or CAR present on the CD8+ T cells. When the antigen can interact with the TCR of the CD8+ T cells the CD8+ T cells can become activated. Examples of various embodiments by which the antigen can be provided in the composition such that it can be recognized by the CD8+ TCR include but are not limited to it the antigen being present in association with MHC-I (or the equivalent presentation in an animal model) on the surface of antigen presenting cells, such as dendritic cells, macrophages or certain activated epithelial cells. Alternatively, the antigen could be in physical association with any other natural or synthesized molecule or other compound, complex, entity, substrate, etc., that would facilitate the recognition of the antigen by the TCR on the CD8+ T cells. For example, the antigen could be complexed to a MHC-I or other suitable molecule for presenting the antigen to the CD8+ TCR, and the MHC-I or other suitable molecule could be in physical association with a substrate, such as a latex bead, plastic surface of any plate, or any other suitable substrate, to facilitate appropriate access of the antigen to the CD8+ T cell TCR such that the antigen is recognized by the CD8+ T cell.

CD8+ T cells may be obtained using routine cell sorting techniques that discriminate and segregate T cells based on T cell surface markers can be used to obtain an isolated population CD8+ T cells for use in the compositions and methods of the invention. For example, a biological sample including blood and/or peripheral blood lymphocytes can be obtained from an individual and CD8+ T cells isolated from the sample using commercially available devices and reagents, thereby obtaining an isolated population of CD8+ T cells. Murine CD8+ T cells may be further characterized and/or isolated on a phenotypic basis via the use of additional cell surface markers such as CD44, L-selectin (CD62L), CD25, CD49d, CD122, , CD27, CD43, CD69, KLRG-1, CXCR3, CCR7, IL-7Ra and KLRG-1. CD8+ T cells may be initially enriched by negatively selecting CD4+, NK1.1+, B220+, CD11b+, TER119+, Gr-1+, CD11c+ and CD19+ cells. Naive CD8+ T cells are characterized as CD44 low, CD62L high, CCR7 high, CD25 low, CD43 low, CD49d low, CD69 low, IL-7Ra high and CD122 low, whereas antigen experienced memory T cells are CD44 high, CD49d high, CD122 high, CD27 high, CD43 high and CXCR3 high. Memory CD8+CD44 high T cells can be further sub-divided into lymphoid-tissue residing Central Memory T cells (CD62L high, CCR7 high) and non-lymphoid tissue residing Effector Memory T cells (CD62L low, CCR7 low) (Klonowski et al. Immunity 2004, 20:551-562). The isolated population of CD8+ T cells can be mixed with the PTPN2 and/or antigen in any suitable container, device, cell culture media, system, etc., and can be cultured in vitro and/or exposed to the one or more antigens, and any other reagent, or cell culture media, in order to expand and/or mature and/or differentiate the T cells to have any of various desired cytotoxic T cell characteristics.

Human CD8+ T-cell types and/or populations can be identified using the phenotypic cell-surface markers CD62L, CCR7, CD27, CD28 and CD45RA or CD45RO (Sallusto F et al. Nature 1999, 401:708-712). As used herein, CD8+ T-cell types and/or populations have the following characteristics or pattern of expression of cell surface markers: Naive T cells are characterized as CD45RA+, CD27+, CD28+, CD62L+ and CCR7+; CD45RO+ Central Memory T cells are CD45RA−, CD27+, CD28+, CD62L+ and CCR7+; CD45RO+ Effector Memory T cells are defined by the lack of expression of these five markers (CD45RA−, CD27−, CD28−, CD62L− and CCR7−); and terminally differentiated Effector Memory CD45RA+ T cells are characterized as CD45RO+, CCR7−, CD27−, CD28−, CD62L−. Terminally differentiated Effector Memory cells further up-regulate markers such as CD57, KLRG1, CX3CR1 and exhibit strong cytotoxic properties characterized by their ability to produce high levels of Granzyme A and B, Perforin and IFNγ. Therefore, various populations of T cells can be separated from other cells and/or from each other based on their expression or lack of expression of these markers. In this manner, the invention provides methods of separating different populations of CD8+ T cells and also separated or isolated populations of CD8+ T cells. The CD8+ T cell types described herein may also be isolated by any other suitable method known in the art; for example, if a particular antigen or antigens are used to produce antigen-specific CD8+ T cells, those cells can be separated or isolated from other cells by affinity purification using that antigen or antigens; appropriate protocols are known in the art.

Different CD8+ T cell types can also exhibit particular functions, including, for example: secretion of IFN-γ; secretion of IL-2; production of Granzyme B; expression of FasL and expression of CD 107. However, while the expression pattern of cell surface markers is considered diagnostic of each particular CD8+ T cell type and/or population as described herein, the functional attributes of each cell type and/or population may vary depending on the amount of stimulation the cell(s) has or have received.

Effector functions or properties of T cells can be determined by the effector molecules that they release in response to specific binding of their T-cell receptor with antigen:MHC complex on the target cell, or in the case of CAR T-cells interaction of the chimeric antigen receptor, e.g. scFv, with the antigen expressed on the target cell. Cytotoxic effector molecules that can be released by cytotoxic CD8+ T cells include perforin, granzymes A and B, granulysin and Fas ligand. Generally, upon degranulation, perforin inserts itself into the target cell's plasma membrane, forming a pore, granzymes are serine proteases which can trigger apoptosis (a form of cell death), granulysin induces apoptosis in target cells, and Fas ligand can also induce apoptosis. Typically, these cytotoxic effector molecules are stored in lytic granules in the cell prior to release. Other effector molecules that can be released by cytotoxic T cells include IFN-γ, TNF-β and TNF-α. IFN-γ can inhibit viral replication and activate macrophages, while TNF-β and TNF-α can participate in macrophage activation and in killing target cells. In any method of the invention, before administration or reintroduction of the cells contacted with a PTPN2 inhibitor, those cells will be assessed for their cytotoxic activity by flow cytometry using fluorochrome-conjugated antibodies against surface and intracellular markers that specify cytotoxic effector T cells including Granzyme A and B, Perforin and IFNγ.

An activated T cell is a cell that is no longer in GO phase, and begins to produce one or more cytotoxins, cytokines and/or other membrane-associated markers characteristic of the cell type (e.g., CD8+) as described herein and is capable of recognizing and binding any target cell that displays the particular peptide:MHC complex or antigen alone on its surface and releasing its effector molecules.

The methods of the invention that promote the differentiation of T cells into a population of cytotoxic T cells lead to a statistically significant increase in the population of cytotoxic T cells. A population is increased when the cells are present in an amount which is at least 10%, 20%, 30%, 40%, 50%, 60%, 70%, 80%, 90%, 95%, or 100% higher in comparison to an appropriate control such as, for example, the size of the population prior to treatment with a method of the invention. The cytotoxic CD8+ T cell effector function is increased when cells have a function which is at least 10%, 20%, 30%, 40%, 50%, 60%, 70%, 80%, 90%, 95%, or 100% higher, than an appropriate control, such as, for example, the performance of a sample of cells in a particular assay in the absence of a particular event or condition. Where appropriate, in vivo function or the presence of a cell population in vivo may be measured using cells isolated from a subject in in vitro assays.

An “enriched” or “purified” population of cells is an increase in the ratio of particular cells to other cells, for example, in comparison to the cells as found in a subject's body, or in comparison to the ratio prior to exposure to a PTPN2 inhibitor. In some embodiments, in an enriched or purified population of cells, the particular cells include at least 20%, 30%, 40%, 50%, 60%, 70%, 75%, 80%, 90%, 95% or 99% of the total cell population. A population of cells may be defined by one or more cell surface markers and/or properties.

CD8+ T cells exposed to, or contacted with, a PTPN2 inhibitor that exhibit at least one property of a cytotoxic T cell as described herein, upon administration to the subject, elicit a cytotoxic T cell response to a tumour cell. Preferably, that CTL response to a tumour cell is effective in causing cell death, such as lysis, of tumour cells having the targeted antigen. CD8+ T cells exposed to, or contacted with, a PTPN2 inhibitor can be administered to the subject by any method including, for example, injection, infusion, deposition, implantation, oral ingestion, or topical administration, or any combination thereof. Injections can be, e.g., intravenous, intramuscular, intradermal, subcutaneous or intraperitoneal. Single or multiple doses can be administered over a given time period, depending upon the cancer, the severity thereof and the overall health of the subject, as can be determined by one skilled in the art without undue experimentation. The injections can be given at multiple locations. Administration of the CD8+ T cells can be alone or in combination with other therapeutic agents. Each dose can include about 10×10³ CD8+ T cells, 20×10³ cells, 50×10³ cells, 100×10³ cells, 200×10³ cells, 500×10³ cells, 1×10⁶ cells, 2×10⁶ cells, 20×10⁶ cells, 50×10⁶ cells, 100×10⁶ cells, 200×10⁶, 500×10⁶, 1×10⁹ cells, 2×10⁹ cells, 5×10⁹ cells, 10×10⁹ cells, and the like. Administration frequency can be, for example, once per week, twice per week, once every two weeks, once every three weeks, once every four weeks, once per month, once every two months, once every three months, once every four months, once every five months, once every six months, and so on. The total number of days where administration occurs can be one day, on 2 days, or on 3, 4, 5, 6, 7, 8, 9, 10, 11, 12, 13, 14, 15, 16, 17, 18, 19, or 20 days, and so on. It is understood that any given administration might involve two or more injections on the same day. For administration, at least 20%, at least 30%, at least 40%, at least 50%, at least 60%, at least 70%, at least 80%, at least 90%, at least 95%, at least 99%, of the CD8+ T cells that are administered exhibit at least one property of a cytotoxic T cell.

It will be clearly understood that, although this specification refers specifically to applications in humans, the invention is also useful for veterinary purposes. Thus in all aspects the invention is useful for domestic animals such as cattle, sheep, horses and poultry; for companion animals such as cats and dogs; and for zoo animals. Therefore, the general term “subject” or “subject to be/being treated” is understood to include all animals (such as humans, apes, dogs, cats, horses, and cows) that require an enhanced immune response, for example subjects having cancer.

As used herein, the term “ex vivo” or “ex vivo therapy” refers to a therapy where cells are obtained from a patient or a suitable alternate source, such as, a suitable allogenic donor, and are modified, such that the modified cells can be used to treat a disease which will be improved by the therapeutic benefit produced by the modified cells. Treatment includes the administration or re-introduction of the modified cells into the patient. A benefit of ex vivo therapy is the ability to provide the patient the benefit of the treatment, without exposing the patient to undesired collateral effects from the treatment.

The term “administered” means administration of a therapeutically effective dose of the aforementioned composition including the respective cells to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

Subjects requiring treatment include those already having a benign, pre-cancerous, or non-metastatic tumour as well as those in which the occurrence or recurrence of cancer is to be prevented. Subjects may have metastatic cells, including metastatic cells present in the ascites fluid and/or lymph node.

The objective or outcome of treatment may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

The method is particularly useful for extending time to disease progression.

The method is particularly useful for extending survival of the human, including overall survival as well as progression free survival.

The method is particularly useful for providing a complete response to therapy whereby all signs of cancer in response to treatment have disappeared. This does not always mean the cancer has been cured.

The method is particularly useful for providing a partial response to therapy whereby there has been a decrease in the size of one or more tumours or lesions, or in the extent of cancer in the body, in response to treatment.

The objective or outcome of treatment may be any one or more of the following:
to reduce the number of cancer cells;
reduce the primary tumour size;
inhibit (i.e., slow to some extent and preferably stop) cancer cell infiltration into peripheral organs;
inhibit (i.e., slow to some extent and preferably stop) tumour metastasis;
inhibit, to some extent, tumour growth;
relieve to some extent one or more of the symptoms associated with the disorder.

In one embodiment, animals requiring treatment include those having a benign, pre-cancerous, non-metastatic tumour.

In one embodiment, the cancer is pre-cancerous or pre-neoplastic.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain. The secondary cancer may be detected in the ascites fluid and/or lymph nodes.

In one embodiment, the cancer may be substantially undetectable.

“Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer is pre-cancerous or pre-neoplastic.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain.

In one embodiment, the cancer expresses the cell surface tumour antigen Her-2.

An example of a cancer that expresses the cell surface tumour antigen Her-2 is a sarcoma.

Other examples of cancer include blastoma (including medulloblastoma and retinoblastoma), sarcoma (including liposarcoma and synovial cell sarcoma), neuroendocrine tumours (including carcinoid tumours, gastrinoma, and islet cell cancer), mesothelioma, schwannoma (including acoustic neuroma), meningioma, adenocarcinoma, melanoma, leukemia or lymphoid malignancies, lung cancer including small-cell lung cancer (SCLC), non-small cell lung cancer (NSCLC), adenocarcinoma of the lung and squamous carcinoma of the lung, epidermoid lung cancer, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumours of the biliary tract, as well as head and neck cancer.

Pre-neoplastic, neoplastic and metastatic diseases are particular examples to which the methods of the invention may be applied. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. B-cell, mixed cell, null-cell, T-cell, T-cell chronic, HTLV-Ilassociated, lymphangiosarcoma, lymphocytic acute, lymphocytic chronic, mast-cell and myeloid), leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour.

It will be understood that the invention disclosed and defined in this specification extends to all alternative combinations of two or more of the individual features mentioned or evident from the text or drawings. All of these different combinations constitute various alternative aspects of the invention.

### Example 1

Mice

Ptpn^{2fl/fl} (C57BL6), Lck-Cre;Ptpn2^{fl/fl} (C57BL6) and RIP-mOVA (C57BL6) were maintained on a 12 h light-dark cycle in a temperature-controlled high barrier facility with free access to food and water. 6-10 week old female recipient mice and 3-6 week old male or female donor mice were used for adoptive transfers. For ex-vivo experiments either male or female mice were used; Ptpn2^{fl/fl} and Lck-Cre;Ptpn2^{fl/fl} or corresponding OT-1 mice were sex-matched. Ptpn2^{fl/fl} and Lck-Cre;Ptpn2^{fl/fl} mice and the corresponding OT-I TCR transgenic mice were described previously (Weide F W (2013) Nature Commun In press.). RIP-mOVA transgenic mice (Kurts C, et al. (1996) Constitutive class I-restricted exogenous presentation of self antigens in vivo. The Journal of Experimental Medicine 184(3):923-930.) were a gift from Bill Heath (University of Melbourne, Australia).

Materials

Recombinant mouse IL-2 was purchased from PeproTech. SIINFEKL peptide was purchased from JPT Peptide Technologies. Hamster α-mouse CD3ε (145-2C11), α-mouse CD28E (37.51) and the FITC-Annexin V Apoptosis Detection Kit I were purchased from BD Biosciences. The FoxP3 Staining Buffer Set and the Cell Stimulation Cocktail (plus protein transport inhibitors) were purchased from eBiosciences. Fetal bovine serum (HyClone) was purchased from Thermo Scientific and Dulbecco-Phosphate Buffered Saline (D-PBS) and 1640 RPMI from Invitrogen.

Flow Cytometry

Single cell suspensions from freshly dissected LNs were processed for flow cytometry as described previously (Weide F W (2013) Nature Commun In press.). For surface staining, cells (1×10⁶/10 μl) were resuspended in D-PBS/2% FBS and stained in 96-well microtiter plates (Falcon, BD Biosciences) for 20 minutes on ice. For sorting, cells were stained in 15 ml Falcon tubes (BD Biosciences) for 30 minutes on ice. Cells were washed and resuspended in D-PBS/2% FBS and analysed using a LSRII (BD Biosciences) or purified using an Influx sorter (BD Biosciences). Purified CD8+CD62L^{hi}CD44^{lo}T cells were routinely tested for purity (>99%). Data was analysed using FlowJo7 (Tree Star Inc.) software.

Antibodies

The following antibodies from BD Pharmingen (San Jose, Calif.) were used for staining: Fluorescein-isothiocyanate (FITC)-conjugated or BD Horizon™ V450-conjugated α-CD44 (IM7), phycoerythrin (PE)-conjugated or allophycocyanin (APC)-conjugated α-CD62L (MEL-14), Pacific Blue-conjugated or Alexa Fluor 647-conjugated α-CD8 (53-6.7), PE-conjugated α-CD25 (PC61), PE-conjugated TCR-Vα2 (B20.1), PE-conjugated TCR-Vβ5.1/5.2 (MR9-4) and PE-cyanine dye 7 (Cy7) α-CD69 (H1.2F3). PE-conjugated α-IFNγ (XMG1.2) and FITC-conjugated granzyme B (GB11) were purchased from Biolegend.

Assessment of CTL-Activity

Naive CD8⁺ (CD62LhiCD44^{lo}) LN T cells (20×10⁵) from 3-4 week old OT-1 mice were purified by FACS and intravenously injected into the tail vein of RIP-mOVA mice. At day 9 post-transfer pancreatic LNs were harvested and homogenised. Cells were resuspened in 1640 RPMI complete and stimulated with the Cell Stimulation Cocktail [containing phorbol 12-myristate 13-acetate (PMA), lonomcyin, Brefeldin A and Monensin)] for 5 h at 37° C. Cells were fixed and permeabilised with the FoxP3 Staining Buffer Set according to the manufacturers' instructions. Cells were stained with fluorochrome-conjugated antibodies against CD8, TCR-Vα2, TCR-V35, IFNγ and granzyme Band analysed by flow cytrometry.

Disease Assessment and Histology

Glycosuria in RIP-mOVA mice was monitored using Diastix (Bayer) and blood glucose levels determined using an Accu-Check glucometer (Roche). Mice were scored as diabetic after two positive readings (urine glucose ≥55 mmol/I) two days apart. Pancreata were harvested and fixed with formalin and embedded in paraffin. Pancreata sections were stained for insulin or glucagon as described previously (Merry T L, et al. (2013) High fat fed obese Gpx1-deficient mice exhibit defective insulin secretion but protection from hepatic steatosis and liver damage. Antioxidants & redox signaling). Blind histological analysis of hematoxylin and eosin-stained (H&E) pancreatic sections was performed to determine the degree of insulitis (grades 0-4). Grade 0 represents no infiltrate, grade 1 periductal accumulation of mononuclear cells, grade 2 circumferential accumulation of mononuclear cells, grade 3 intra-islet infiltration, and grade 4 represents severe structural derangement and complete loss of beta cells. All islets were scored from three sections 100 μm apart. The proportion of islets with grades 0-4 respectively was determined and expressed as a percentage of the total number of islets scored.

Adoptive Transfers into RIP-mOVA Mice

For the induction of type I diabetes in RIP-mOVA mice, 2.5×10⁵, 8×10⁵ or 20×10⁵ CD8⁺CD62L^{hi}CD44^{lo} naive LN T cells from 3-4 week old OT-1 mice were purified by FACS and intravenously injected into the tail vein of RIP-mOVA mice. CD8+OT-I T cells donor cells were identified by gating for CD8+ and TCR-Vα2+TCR-V35+ cells. For assessing the in vivo proliferative response of CD8+CD62L^{hi}CD44^{lo} naïve LN T cells from OT-1 mice, 2×10⁶ CFSE stained cells from 3-4 week old mice were transferred intravenously into the tail vein of RIP-mOVA mice. Proliferating donor CD8+OT-I T cells were identified by gating for CD8+ and TCR-Vα2+CFSE+ cells.

In Vitro Stimulation

CFSE stained or unstained CD8+CD62L^{hi}CD44^{lo} naïve LN T cells (1×10⁵/well) from TCR-transgenic OT-1 or non-transgenic mice were stimulated in 96-well round-bottom plates with 0.1 μg/ml SIINFEKL for 36h or with plate-bound α-CD3 (10 μg/ml) and α-CD28 (5 μg/ml) for 48h. To determine the activation status cells were harvested, stained with fluorochrome-conjugated antibodies against α-CD25, α-CD44 and α-CD69 and analysed by flow cytometry.

In Vitro Apoptosis

CD8+CD62L^{hi}CD44^{lo}naive LN T cells (2×10⁵/well) from TCR-transgenic OT-1 mice were stimulated in 96-well round-bottom plates with 1 μg/ml SIINFEKL for 18h. Cells were harvested and stained with the FITC-Annexin V Apoptosis Detection Kit and analysed by flow cytometry. For quantification Calibrite Beads (BD Biosciences) were added to the wells before cells were harvested. Cells were classified as propidium iodide (PI)⁻ Annexin V⁻ (live), propidium iodide (PI)⁻ Annexin V⁺ (apoptotic) and propidium iodide (PI)⁺ Annexin V⁺ (necrotic).

In Vitro Generation of Effector/Memory T Cells

CD8⁺CD62L^{hi}CD44^{lo}naive LN T cells (1×10⁵/well) were stimulated in 96-well round-bottom plates with plate-bound α-CD3 (10 μg/ml) and α-CD28 (5 μg/ml) as described previously (Weide FW (2013). Nature Commun In press.). 48h post-stimulation cells were harvested, washed and counted and 0.5×10⁵ cells/well incubated with murine IL-2 (20 ng/ml) in flat-bottom 96-well plates in the absence of α-CD3/α-CD28. At 2 and 4 days post IL-2 administration cells were stained with fluorochrome-conjugated antibodies against CD44 and CD62L and the generation of effector/memory T cells quantified with Calibrite Beads (BD Biosciences) by flow cytometry.

CFSE Staining

For the assessment of T cell proliferation by CFSE (Invitrogen-Molecular Probes) dilution, lymphocytes (1×10⁷/m1) were resuspended in D-PBS supplemented with 0.1% (v/v) BSA. CFSE was added at a final concentration of 5 μM and cells were incubated for 5 min at room temperature. Cells were then washed three times with D-PBS supplemented with 10% (v/v) FBS before adoptive transfer or in vitro stimulation.

Statistical Analysis

Statistical analysis was performed with Graphpad Prism software using the non-parametric using 2-tailed Mann-Whitney U Test. Statistical analyses on Kaplan-Meier estimates were performed using a Log-rank (Mantel-Cox) test with one degree of freedom. For both tests p<0.05 was considered as significant.

Animal Ethics

All experiments were performed in accordance with the NHMRC Australian Code of Practice for the Care and Use of Animals. All protocols were approved by the Monash University School of Biomedical Sciences Animal Ethics Committee (Ethics number: MARP/2012/124).

### Example 2

PTPN2-Deficient OT-I CD8+ T Cells Promote Type 1 Diabetes in RIP-mOVA Mice in the Absence of CD4+ Help.

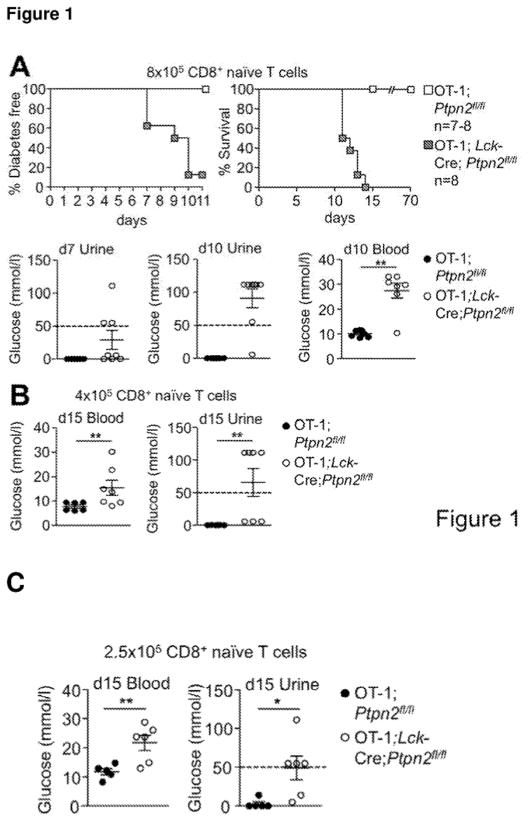

To examine the consequence of PTPN2-deficiency on CD8+ T cell cross-priming and the development of autoimmune diabetes we took advantage of RIPmOVA mice (Kurts C, et al. (1996) The Journal of Experimental Medicine 184(3):923-930.). RIP-mOVA mice express a membrane bound form of ovalbumin (OVA) in the β cells of the pancreas and in the renal proximal tubular cells of the kidney (Kurts C, et al. (1996) The Journal of Experimental Medicine 184(3):923-930.). However, thymic expression of OVA as ‘self’ in RIP-mOVA mice results in tolerance. OT-I mice express the Vα2/Vβ5 TCR that is specific for the OVA peptide 2⁵⁷SIINFEKL²⁶⁴ (presented in the context of K^{b}class I MHC) selecting for CD8+ single positive thymocytes (Hogquist K A, et al. (1994) Cell 76(1):17-27). The adoptive transfer of naive OT-I CD8+ T cells alone into RIP-mOVA mice results in cross-presentation and the deletion of ‘autoreactive’ OT-I CD8+ T cells. This process of cross-tolerance is impaired by the co-transfer of OVA-specific CD4+ T cells, resulting in cross-priming and p cell destruction and type 1 diabetes. Although early studies showed that loss of tolerance and type 1 diabetes can also be triggered by the transfer of high numbers of OT-I donor T cells on a Rag1^{-/-} background, subsequent studies utilising OT-I T cells on a C57BL/6 background have found that naive OT-I CD8+ T cells on their own do not result in type 1 diabetes [even after the transfer of 10×10⁶ OT-I CD8+ T cells]. To assess the impact of PTPN2-deficiency on CD8+ T cell cross priming and the development of type 1 diabetes, Ptpn2^{fl/fl} and Lck-Cre; Ptpn2⁴′ mice were bred onto the OT-I TCR transgenic background. Naive (CD44^{lo}CD62L^{hi}) OT-1;Ptpn2^{fl/fl} and OT-I;Lck-Cre;Ptpn2^{fl/fl} CD8+ T cells were transferred into RIP-mOVA recipients and the incidence of diabetes (urine glucose >50 mM) and survival monitored. As expected mice receiving 2.5-8×10⁵ OT-1; Ptpn2^{fl/fl} control CD8+ T cells did not exhibit any signs of diabetes/morbidity even after 70 days (FIG. 1), consistent with the induction of peripheral tolerance. In contrast, mice receiving 8×10⁵ OT-I;Lck-Cre;Ptpn2^{fl/fl} CD8+ T cells exhibited elevated urine glucose levels and diabetes as early as seven days post-transfer and 100% of mice succumbed to severe dehydration within fourteen days of adoptive transfer (FIG. 1a).

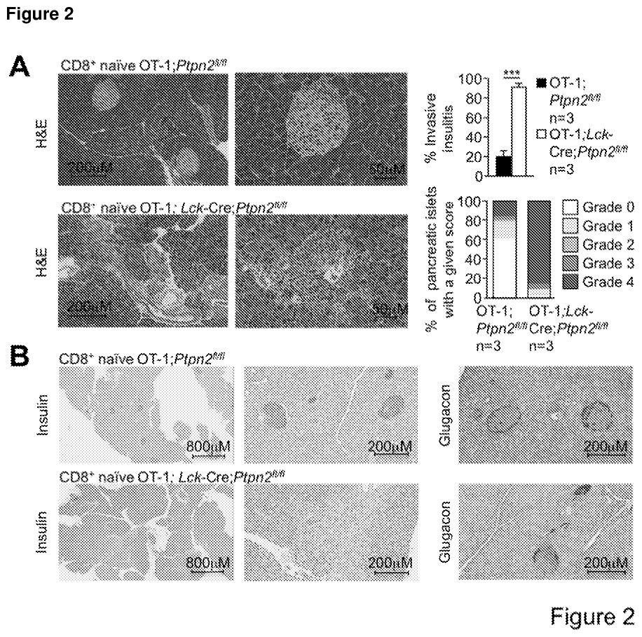

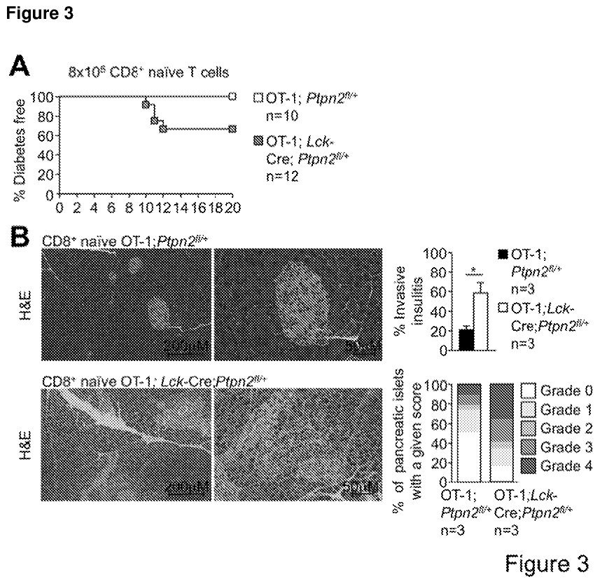

Similarly four of seven mice receiving 4×10⁵ OT-1;Lck-Cre;Ptpn2^{fl/fl} CD8+ T cells exhibited hyperglycemia and glycosuria/diabetes by 15 days post-transfer (FIG. 1b), whereas four out of six mice receiving 2.5×10⁵ OT-I;Lck-Cre;Ptpn2^{fl/fl} CD8+ T cells exhibited glycosuria (FIG. 1c). Histological analysis [hematoxylin and eosin (H&E) staining] at eleven days post-transfer revealed that the development of diabetes in mice receiving 8×10⁵ OT-I;Lck-Cre;Ptpn2^{fl/fl} CD8+ T cells was accompanied by destructive insulitis, with numerous pyknotic nuclei present consistent with cellular apoptosis, near obliterative lesions in remnant islets and striking immune cell infiltration (FIG. 2a). Immunohistochemical analyses revealed markedly decreased insulin staining with diminished glucagon staining in islet remnants, consistent with p cell and islet destruction (FIG. 2b). In contrast, mice receiving OT-1;Ptpn2^{fl/fl} control CD8+ T cells showed destructive insulitis in only 20% of islets with the remaining exhibiting either peri-insulitis, or no infiltration (FIG. 2). Given the association of PTPN2 SNPs with type 1 diabetes we also assessed the impact of Ptpn2 heterozygosity on CD8+ T cell cross priming and the development of type 1 diabetes. 8×10⁵ OT-1;Ptpn2^{fl/fl} or OT-I;Lck-Cre;Ptpn2^{fl/+} CD8+ T cells were transferred into RIP-mOVA recipients and the incidence of diabetes monitored. Four out of twelve mice receiving OT-I;Lck-Cre;Ptpn2^{fl/+} CD8+ T cells exhibited marked glycosuria/diabetes by twenty days post-transfer [p<0.05; Long Rank (Mantel-Cox) test] (FIG. 3). A histological assessment at twenty days post-transfer in mice that received OT-I;Lck-Cre;Ptpn2^{fl/+} CD8+ T cells and developed diabetes revealed that this was associated with destructive insulitis in greater than 50% of islets examined, as compared to 20% in mice reeciving OT-I;Ptpn2^{fl/+} CD8+ T cells (FIG. 3). Therefore, homozygous or heterozygous PTPN2-deficiency in CD8+ T cell leads a loss of tolerance and results in p cell destruction and the development of autoimmune diabetes in the absence of CD4+ help.

The Initial Proliferation of OT-I CD8+ T Cells in RIP-mOVA Mice is not Affected by PTPN2-Deficiency.

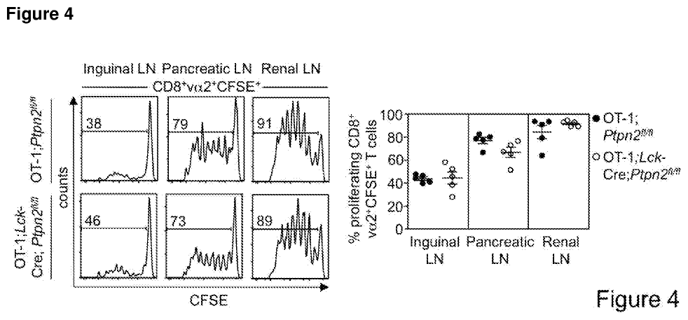

When OT-I CD8+ T cells are cross-presented in the draining lymph nodes of the pancreas and kidneys in RIP-mOVA mice, they undergo initial expansion, after which they become tolerarised and are deleted. We first determined whether the promotion of autoimmune diabetes associated with the adoptive transfer of PTPN2-deficient CD8+ T cells may be linked to enhanced proliferative responses. CFSE-labeled naive (CD44^{lo}CD62L^{hi}) OT-1;Ptpn2^{fl/fl} and OTI; Lck-Cre;Ptpn2^{fl/fl} CD8+ T cells (2×10⁶) were transferred into RIP-mOVA recipients and cellular proliferation assessed after three days by CFSE dilution (FIG. 4). Cellular proliferation was assessed in the T cells residing in pancreatic, renal and inguinal LNs. No significant differences in cellular proliferation were evident with OT-1;Ptpn2^{fl/fl} and OT-I;Lck-Cre;Ptpn2^{fl/fl} CD8+ T cells in the LNs draining the pancreas and kidney; OT-1;Ptpn2^{fl/fl} and OT-I;Lck-Cre;Ptpn2^{fl/fl} CD8+ T cells underwent a similar number of divisions after three days (FIG. 4). In keeping with OVA expression being restricted to the pancreas and kidney we did not detect any overt OT-I CD8+ T cell proliferation in inguinal LNs (FIG. 4). Therefore, the loss of tolerance and development of autoimmune diabetes associated with PTPN2 deficiency cannot be ascribed to enhanced proliferative responses.

PTPN2-Deficiency Results OT-I CD8+ T Cell Differentiation in RIP-mOVA Mice.

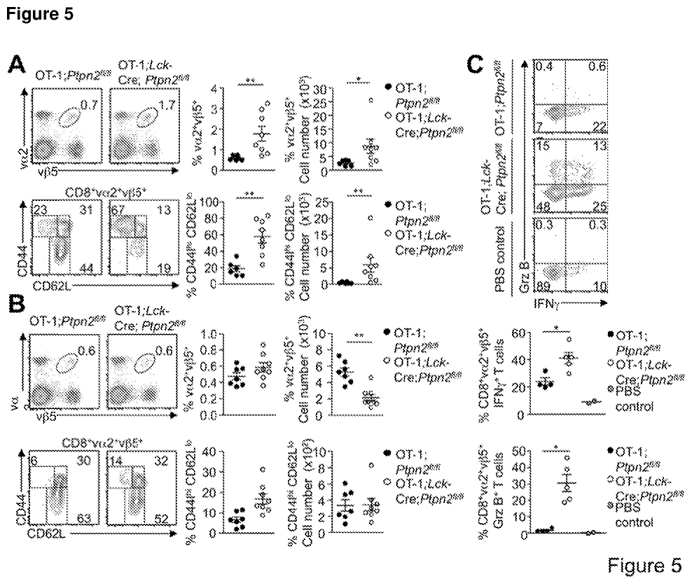

Next we determined whether the autoimmune diabetes evident after the transfer of OT-I;Lck-Cre;Ptpn2^{fl/fl} CD8+ T cells into RIP-mOVA mice might be associated with the enhanced generation of CD8+ CTLs in vivo. Naive (CD44^{lo}CD62L^{hi}) OT-1;Ptpn2^{fl/fl} and OT-I;Lck-Cre;Ptpn2^{fl/fl} CD8+ T cells (8×10⁵) were transferred into RIP-mOVA and the generation of CD44^{hi}CD62L^{lo} effector/memory T cells assessed in the pancreatic and inguinal LNs. After eleven days we found that PTPN2-deficiency increased both the total number of OT-I CD8+ T cells and the number of CD44^{hi}CD62L^{lo} OT-I CD8+ effector/memory T cells in the pancreatic LNs, but not in the inguinal LNs (FIG. 5a-b). Interestingly, in the inguinal LNs, the total number of PTPN2-deficient OT-I CD8+ T cells decreased, probably as a result of OT-I CD8+ T cell homing to the pancreas (FIG. 5b). These results are consistent with PTPN2-deficiency enhancing the generation of OT-I CD8+ effector/memory T cells after cross-priming in pancreatic LNs. To determine whether the OT-I;Lck-Cre;Ptpn2^{fl/fl} CD44^{hi}CD62L^{lo}CD8+ T cells exhibited CTL activity, we measured interferon (IFN)γ and granzyme B levels, two important proteins for CD8⁺ T cell killing. OT-I CD44^{hi}CD62L^{lo}CD8+ T cells were isolated from the pancreatic LNs of RIP-mOVA mice that had received naive OT-I;Ptpn2^{fl/fl} versus OT-I;Lck-Cre; Ptpn2^{fl/fl} CD8+ T cells and IFNγ and granzyme B levels assessed by intracellular staining after a brief re-stimulation with PMA/lonomycin (FIG. 5c). PTPN2-deficiency resulted in the generation of CD8+ CTLs (FIG. 5c). IFNγ levels were elevated by 2 fold in OT-I;Lck-Cre;Ptpn2^{fl/fl} versus OT-I;Ptpn2^{fl/fl} T cells (FIG. 5c). Moreover, granzyme B was evident in OT-I;Lck-Cre;Ptpn2^{fl/fl} CD8+ T cells, but not in OT-I; Ptpn2^{fl/fl} control T cells (FIG. 5c), consistent with the latter being tolerised. Taken together, these results are consistent with PTPN2-deficiency promoting CD8+ T cell differentiation and the generation of CTLs after the cross-presentation of self-antigen.

PTPN2-Deficiency Enhances Antigen-Induced OT-I CD8+ T Cell Activation and IL-2-Mediated Differentiation In Vitro

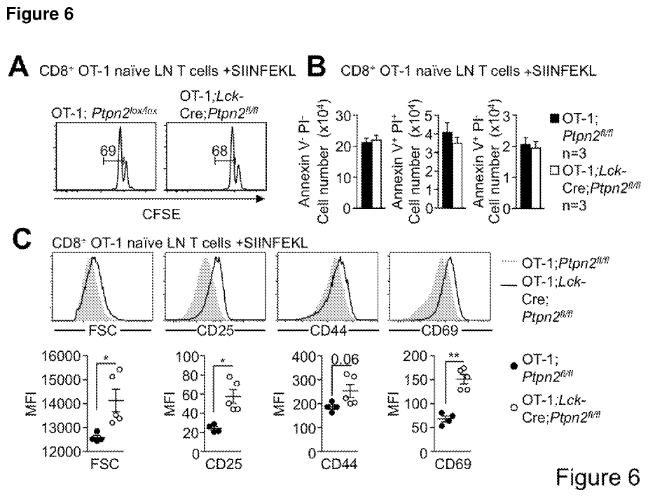

PTPN2-deficiency lowers the threshold for TCR-induced proliferation so that naive CD8+OT-I T cells undergo enhanced proliferation when challenged with low concentrations of cognate peptide antigen SIINFEKL, or altered peptide ligands with suboptimal TCR affinity. However, PTPN2-deficiency does not enhance OT-I CD8+ T cell proliferation in response to high concentrations SIINFEKL. To determine whether PTPN2 deficiency might enhance OT-I CD8+ T cell activation independent of proliferation we stimulated OT-1;Ptpn2^{fl/fl} and OT-I;Lck-Cre;Ptpn2^{fl/fl} naive (CD44^{lo}) CD8+OT-I T cells with saturating concentration of SIINFEKL (0.1 μg/ml) for 36 h (FIG. 6). Since CD8+ T cell responses induced by peptide presented by anchored class I MHC can be ascribed predominantly to eluted peptide self-presented by T cells, we added peptide directly to the culture supernatant. PTPN2-deficiency did not alter the proliferation OT-I CD8+ T cells (as assessed by FSE dilution after 36 h) challenged with high concentrations of SIINFEKL (FIG. 6a). Moreover, PTPN2 deficiency did not alter OT-I CD8+ T cell survival (as assessed by Annexin V staining) (FIG. 6b). On the other hand, OT-I CD8+ T cell activation, as assessed by monitoring the cell surface expression of CD69 and the IL-2 receptor a (CD25)-subunit, as well as blast formation (cell size) by flow cytometry, was significantly enhanced by PTPN2 deficiency (FIG. 6c). Moreover, cell surface CD44 expression levels were increased (FIG. 6c), consistent with the CD8+ T cells undergoing differentiation.

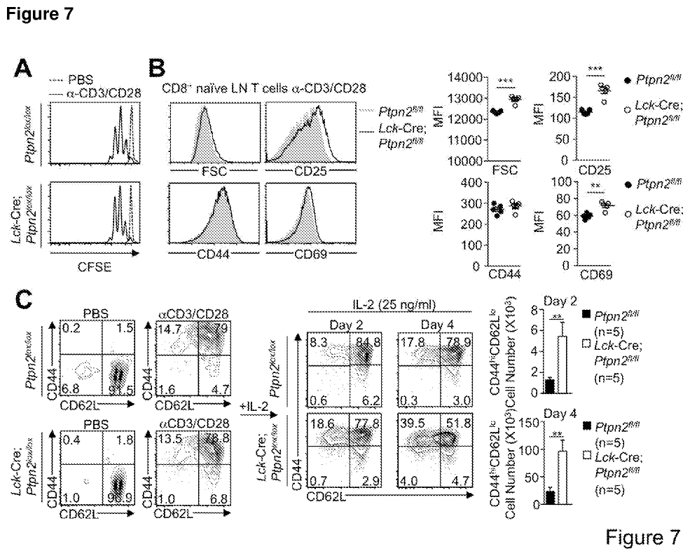

Given that PTPN2-deficiency enhanced TCR-induced CD25 expression and previous studies have highlighted the capacity of PTPN2 to attenuate IL-2-induced STAT5 signaling, we assessed the impact of PTPN2-deficiency on the IL-2-induced differentiation of CD8+ effector T cells. When naive CD8+ T cells are cultured with a strong stimulus in conjunction with CD28 co-stimulation and then removed from this stimulus and incubated with IL-2, they undergo differentiation into CD44^{hi}CD62L^{lo} T cells and acquire CTL activity characterised by the expression granzyme B and IFNγ. Naive (CD44^{lo}CD62L^{hi}) CD8+ T cells from Ptpn2^{fl/fl} and Lck-Cre; Ptpn2^{fl/fl} mice were stimulated with saturating concentrations of plate bound α-CD3F (10 μg/ml) plus α-CD28 (5 μg/ml) for 48 and then removed from stimulation and cultured in the presence of 20 ng/ml IL-2 for 2-4 days and the cell surface expression of CD44 and CD62L determined (FIG. 7). At these saturating concentrations of α-CD3F/α-CD28, PTPN2 deficiency neither affected proliferation (as assessed by FSE dilution) nor the increase in cell surface CD44 (FIG. 7a-b), but increased CD25 and CD69, albeit modestly, consistent with enhanced TCR-mediated activation (FIG. 7b). However, PTPN2-deficiency resulted in a striking increase in the number of cells that were CD44^{hi}CD62L^{lo} after IL-2 stimulation (FIG. 7c). Taken together these results are consistent with PTPN2-deficiency driving the TCR and IL-2-mediated differentiation of CD8+ effector T cells.

Early studies using the RIP-mOVA model and ‘high affinity’ TCR transgenic T cells established that tissue-specific antigens can be presented constitutively and selectively to naive CD8+ T cells by DCs residing in the corresponding draining LNs, to limit T cell activation and promote tolerance. Subsequent studies have established this also be true for polyclonal T cells expressing low- to intermediate-affinity TCRs. However, in the absence of help from CD4+ T cells, cross-presented CD8+ T cells undergo clonal deletion to maintain tolerance. Such cross-tolerance may be induced at least in-part by T cell intrinsic mechanisms that tune TCR signaling and limit T cell responses to self. In this study we have used the RIP-mOVA model and OT-I T cells to establish that the phosphatase PTPN2 is instrumental in tolerising CD8+ T cells that have been cross-presented endogenous antigens.

PTPN2 levels are elevated in naive T cells leaving the thymus and that increases in PTPN2 directly correlate with TCR affinity (as monitored by CD5 levels), so that higher affinity T cells, responding more robustly to self-antigen in the context of lymphopenia, have increased PTPN2. In the absence of PTPN2 naive CD8+ T cells undergo fast-paced TCR-mediated proliferation in a lymphopenic environment, acquire the characteristics of antigen-experienced effector cells, and promote the development of autoimmunity. In this study we found that PTPN2-deficiency allowed CD8+ T cells cross-primed by p cell self-antigens to escape tolerance and acquire CTL activity and thus promote p cell destruction and the development of type 1 diabetes. In the absence of PTPN2, OT-I CD8+ T cells alone were able to promote diabetes in RIP-mOVA mice, this occurred even when very low numbers of OT-I CD8+ T cells (2.5×10⁵) were transferred. Importantly, this was not associated with enhanced OT-I CD8+ T cell proliferation in the draining pancreatic LNs, but rather with the increased differentiation of naive T cells into CD44^{hi}CD62L^{lo} CTLs. Our ex vivo studies suggest that the enhanced generation of CD44^{hi}CD62L^{lo} CTLs might be ascribed to enhanced TCR- and IL-2-induced responses. In the context of an infection, IL-2 signaling in CD8+ T cells is essential for the development of terminally-differentiated CTLs. Here we have shown that under saturating conditions, when differences in TCR-induced proliferation are not evident, but TCR-induced CD25 levels are elevated, PTPN2 exacerbates the IL-2-induced generation of CD44^{hi}CD62L^{lo} CD8+ T cells. Beyond promoting TCR-induced T cell activation, PTPN2 also has the capacity to dephosphorylate STAT family members including STAT5, STAT3 and STAT1, which mediate IL-2, IL-6 and IFNγ-induced signaling respectively in CD8+ T cells. Such cytokines play crucial roles in regulating CD8+ T cell expansion, differentiation and survival. For example, excessive IFNγ has recently been shown to cause the increased accumulation of short-lived effector CD8+ T cells in autoimmune-prone Roquin gain-of-function mice. Therefore, PTPN2-deficiency could at least in part contribute to development of autoimmunity-linked CTLs by directly promoting cytokine signaling including IL-2 signaling.

Our results highlight the potential for alterations in TCR tuning as a consequence of alterations in negative regulators such as PTPN2 to subvert the tolerisation of cross-presented CD8+ T cells to contribute to the development of autoimmunity. The type 1 diabetes-associated PTPN2 SNP [(rs1893217(C)] has been linked with an approximate 40% decrease in PTPN2 message in T cells. Our studies indicate that PTPN2 heterozygosity in OT-I CD8+ T cells is sufficient to abrogate peripheral tolerance induced by cross-presentation in RIP-mOVA mice. Thus, our studies highlight a mechanism by which the PTPN2 SNP may contribute to the development of autoimmunity.

Beyond the contributions to peripheral tolerance, antigen cross-presentation is crucial for the priming of CD8+ cytotoxic T cell responses to pathogens and antigens in tumours. Numerous phase I and II trials have been performed using DC vaccination or adoptive T cell therapy as anti-viral or anti-tumour treatments. Importantly, T cells isolated from human tumours exhibit many of the characteristics of exhausted or tolerised T cells; CD8+ T cells isolated from tumours express inhibitory receptors such as PD-1 and exhibit defective cytokine production.

### Example 3

The following is an exemplary method of producing CAR-T cells using retroviral infection (Cheadle E J et al. J Immunol 2010; 184:1885-1896). This approach to expressing a specific CAR is also relevant for expressing a shRNA or siRNA to reduce the expression of PTPN2 in CAR-T cells or any other T cell type described herein. While this method is designed for application in a murine setting, changes can be made for application in humans based on methodology described in, for example, Themeli, et al. (2013), Nature Biotechnology, 31(1), pp 928 to 933 (including associated online methods) and Tran et al. (2014), Science, 344, pp 641 to 645.

### Day 0—Isolation Of T-Cells

1. To isolate splenocytes, excise spleen from BI/6 mouse
2. Spray EtOH on mouse and utensils (scissors/forceps).
3. Make incision on back of mouse and use fingers to pull back skin. Spleen is on left side, bright burgundy colour.
4. Snip of fat attaching it to abdomen and place in petri dish (non-coated)
5. Add 5 ml Dulbecco's PBS or media to spleen.
6. Open syringe, keeping plunger end sterile. Use this end to mash spleen into a single cell suspension—approx. 1×10⁸ cells/spleen.
7. Place a nylon 70 uM sieve in 50 ml falcon tube and run this mixture through. Fatty tissue will collect in the sieve.
8. Add another 5 ml PBS or media into petri dish to collect remaining cells and flush through sieve >>total of 10 ml splenocytes—RBCs and WBCs.
9. Spin 1250 rpm, 5 min to obtain a pellet.
10. Discard supernatant by use of suction.
11. Resuspend pellet in 3 ml ACK lysis buffer (top shelf—RT). This buffer gets rid of RBCs.
12. Invert and rock back and forth for 4 mins in hand
13. Add 15 ml RPMI (10%+Lot) media.
14. Aliquot 10 ul of this mixture for cell count during spin—keep in filter tip.
15. Spin 1250 rpm, 5 min.
16. Discard supernatant using suction apparatus—pellet shouldn't be red in colour anymore.
17. Add Xml RPMI media to pellet to resuspend at 2.5×10⁷ cells/well.
18. Add cytokines—use filter tips and keep sterile:
   (fresh aliquots in freezer below cell count microscope, working stock in fridge—can be kept at 4° C. for up to 1 mth)
19. Mix.
20. Aliquot 5 ml/well (normal 6-well plate)—2.5×10⁷ cells/ml
21. Label plate with cells (C57 splenocytes), media, name, date, 5×10⁶ cells/ml.
22. Place in incubator (5% CO₂) O/N.

### Cell Count

1. Place 10 μl of cells collected in Step 15. into 96-well plate near microscope.
2. Add 10 μl of tryptan blue to well (dilution factor of 2).
3. Put square coverslip on top of cell count slide (blow breath onto slide first so that coverslip slides onto it well).
4. Aliquot cells onto slide and view under the microscope.
5. Dead cells take up tryptan blue whereas live cells don't.
6. Count cells—if cells appear well spaced out, count bottom 5 squares and multiply by 5 to cover whole grid.

### Retronectin Plates

1. Retronectin (10 μg/ml)
   100 μl aliquot/tube—1 tube/plate—100 μl/10 ml PBS
2. 6-well plates
3. Pipette 1.5 ml/well (can re-use retronectin/PBS).
4. Keep O/N at 4° C. (or 37° C. 2 hrs, if forget to set it up overnight).

### Viral Packaging Lines

1. Harvest and seed out viral packaging lines at 6×10⁶ cells/medium flask containing 25 mls media.

### Day 1

### Viral Packaging Lines

1. Obtain flask containing viral packaging lines from incubator (10% CO₂).
2. Pour supernatant containing viral particles into a 60 ml syringe and filter into a labelled 50 ml falcon tubes—should have a total volume of ˜25 ml of each packaging line.
3. Replace flask with fresh RPMI (10%+Lot) media (25 ml) and put flasks back into incubator (5% C02).

### Retronectin Plates

1. Tilt plate and use suction to remove retronectin.
2. Add 2.5 ml of viral packaging lines supernatant (pLXSN and Erb) to wells.
3. Wrap plates tightly in cling-wrap making sure edges are tightly sealed. Excess cling-wrap can sit on top of plate.
4. Place in centrifuge—make sure plates are balanced correctly and spin at 1200×g, 30 mins, RT (Program 4).

### Harvesting T-Cells

1. Take 6-well plate (5 ml media/well) containing T-cells from incubator (5% C02) and check under microscope.
2. Harvest T-cells by using a plastic pipette to dislodge cells from base of each well. Pipette up and down (3-4 times) and transfer to fresh 50 ml falcon tube labelled ‘AII T cells’. Total volume=30 mls
3. Make sure that each 50 ml falcon tube has no more than 30 ml of solution.
4. Pipette 10 ml Ficoll-Plaque (RT-shelf) on slowest pipette speed gently into the bottom of the falcon tube containing ‘AII T cells’. There should be a gradient at this stage, with the gel sitting at the bottom of the tube.
5. Centrifuge 1800 rpm at acceleration/deceleration 6 for 10 mins.
6. Label fresh 50 ml falcon tube with ‘Live T cells’.
7. Ficoll-plaque separates the live cells from the dead cells. Centrifuging the T cells in the Ficoll-plaque gel gradient will cause the dead cells to remain at the bottom of the gel, and the live cells to sit immediately on top of the gel below the supernatant. Aspirate off as much of the media supernatant—careful not to get too close to the T cells. Remove live T cells into the ‘Live T cells’ falcon tube using a 1 ml pipette.
8. Resuspend live T cells in 10 ml fresh RPMI (10%+Lot) media.
9. Aliquot out 10 ul of cells into filter tip and transfer to 96-well plate (near microscope) for cell count.
10. Split 10 ml volume of resuspended T cells into falcon tubes depending on number of packaging lines—eg. 2×5 ml—LXSN+Erb
11. Spin 50 ml falcon tubes at 1250 rpm, 5 mins (Program 1).
12. Following spin, discard supernatant using suction—be careful not to discard pellet of T cells.
13. Add Xml of viral sup to correctly labelled pellet and resuspend.
14. Add cytokines: (take into account retronectin plate)
15. Pipette out 2.5 ml/well (3 wells/supe type)
16. Wrap plates in cling wrap, as before and spin for 1.5 hrs at 1200×g (Program 4—alter time).
17. Once spin is complete—carefully remove cling-wrap and place plates in incubator (5% C02).

### Day 2

1. Check flasks containing viral packaging lines (5% CO2 incubator).
2. Pour supernatant containing viral particles into a 60 ml syringe and filter into a labelled 50 ml falcon tubes—should have a total volume of −25 ml of each packaging line.
3. Can split cells into large flask during spins to keep lines growing.
   Harvesting T-Cells from Retronectin Plates
4. Check retronectin plates—T cells+pLXSN/Erb supe (3 wells per sample—2× plates) (5% CO2 incubator).
5. Harvest T-cells by using a plastic pipette to dislodge cells from base of each well. Pipette up and down (3-4 times) and transfer to fresh 50 ml falcon tube. Total volume=15 mls of each sample.
6. Can do a PBS wash as well if not all cells removed.
7. Keep these falcon tubes in 5% CO2 incubator until ready to pellet.*
8. To ‘empty’ retronectin plates (from Step 3-4), add 2.5 ml of viral supernatant (from Step 2)—check that aliquoting supernatant into correctly labelled plate.
9. Wrap in cling-wrap, as done on Day 1 and spin 1200×g, 30 mins (Program 4).

### Splitting Cells

10. To flasks containing the packaging lines [pLXSN and Erb]—since viral cells were confluent >>need to split.
11. Add 0.5 ml tryple (stored at 4° C.) (trysin) to flasks and gently rock to make sure cells are covered in tryple.
12. Discard tryple by using suction.
13. Leave flasks for 5-10 mins at RT, or put in incubator (5% C02) to speed up process to remove cells attached to flask.
14. Put 30 ml media (RPMI) into fresh large flask.
15. Once cells are deattached—add 10 ml of RPMI media and pipette up and down to move cells to bottom of flask.
16. Take 1 ml of this and pipette into the new large flask (check labelling)>>1:10 dilution—cells will be confluent in 3-4 days. Need to check and split once over 80% confluent.
17. Place flasks in 5% CO2 incubators.

### *Harvesting T-Cells (Cont.)

18. Spin 50 ml falcon tubes containing T-cells at 1250 rpm, 5 mins (Program 1).
19. Following spin, discard supernatant using suction—be careful not to discard pellet of T cells.
20. Add *7.5 ml of viral supernatant (for 3 wells) to correctly labelled pellet and resuspend gently. Excess supernatant can be discarded.
21. Add cytokines (take into account retronectin plate):

### IL2—15 μl

### IL7—30 μl

22. Pipette out 2.5 ml (T cell-viral complex)/well on retronectin plates (3 wells/plate >>×10⁶ cells/well)
23. Wrap plates in cling wrap, as before and spin for 1.5 hrs at 1200×g (Program 4—alter time).
24. Once spin is complete—carefully remove cling-wrap and place plates in incubator (5% C02) for 4.5 hrs.
25. Harvest T-cells by using a plastic pipette to dislodge cells from base of each well. Pipette up and down (3-4 times) and transfer to fresh 50 ml falcon tube. Total volume=15 mls of each sample.
26. Good to do a PBS wash if doing in vitro assays to collect maximum amount of cells.
27. Spin 50 ml falcon tubes at 1250 rpm, 5 mins (Program 1).
28. Following spin, discard supernatant using suction—be careful not to discard pellet of T cells.
29. Add 15 ml of RPMI media to falcon tubes+cytokines (15 μl IL2 and 30 μl IL7). Give a quick swirl.
30. Aliquot 5 ml/well (3 wells/sample) into a fresh labelled normal 6-well plate.
31. Incubate in 5% CO2 incubator until ready to add more cytokines or split/expand two days later.

### Day 4/5

Check transduced T cells—if not ready to split add another 5 ml fresh RPMI with cytokines to each well (on top of media already in well).
6 wells altogether (3×LXSN+3× Erb)=30 ms required
+30 μl IL2 and 60 μl IL7
   Incubate in 5% CO2 37C incubator until ready to harvest on Day 7-8.

### Day 7-8

Harvest transduced T cells for in vitro/in vivo assays.

### Example 4

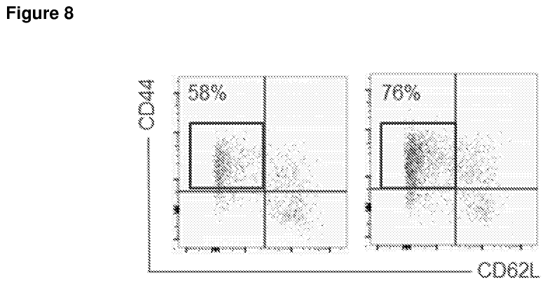

Splenocytes from Lck-Cre;Ptpn2^{fl/fl} (−/−) versus Ptpn2^{fl/fl} (+/+) mice were stimulated and retrovirally co-transduced with scFv-anti-Her-2 and CD28-CD3-ζ ((α-Her-2). After 8 days in culture Her-2 specific Lck-Cre;Ptpn2^{fl/fl} (α-Her-2−/−) versus Ptpn2^{fl/fl} control (α-Her-2+/+) CAR CD8+ T cells were stained with fluorochrome-conjugated antibodies against CD62L versus CD44 and the generation of effector/memory (CD44^{hi}CD62L^{lo}) was monitored by flow cytometry. The results in FIG. 8 show PTPN2-deficiency enhances the acquisition of an effector/memory phenotype in CD8+ CAR T cells ex vivo.

### Example 5

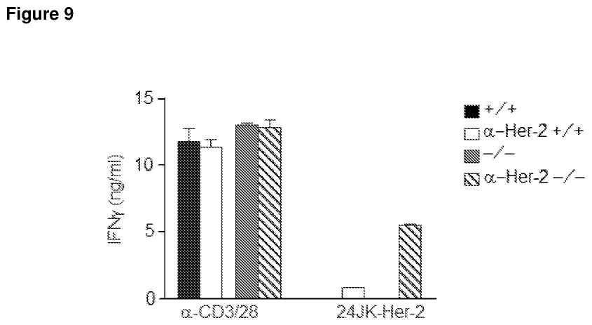

Her-2-specific Ptpn2^{fl/fl} (α-Her-2+/+) versus Lck-Cre;Ptpn2^{fl/fl} CD8⁺ CAR T cells (α-Her-2−/−) or non-transfected Ptpn2^{fl/fl} (+/+) versus Lck-Cre;Ptpn2^{fl/fl} (−/−) CD8⁺ T cells were incubated with Her-2 expressing 24JK sarcoma cells (24JK-Her-2) or plate-bound a-CD3/CD28 (as a non-antigen-specific means by which to activate T cells). T cell activation was assessed by monitoring for secreted IFNγ by ELISA. The results in FIG. 9 show PTPN2-deficiency enhances CD8+ CAR T cell activation ex vivo.

### Example 6

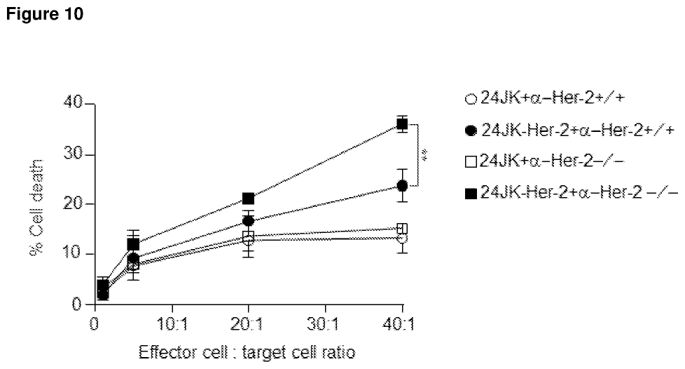

Her-2-specific Ptpn2^{fl/fl} (α-Her-2+/+) versus Lck-Cre;Ptpn2^{fl/fl} CAR CD8⁺ T cells (α-Her-2−/−) CD8+ T cells were incubated with chromium (Cr⁵¹) labelled 24JK-Her-2 sarcoma cells or Her-2 negative 24JK sarcoma cells and Cr51 release measured four hours later. The results in FIG. 10 show PTPN2-deficiency enhances CAR T cell CTL activity ex vivo.

### Example 7

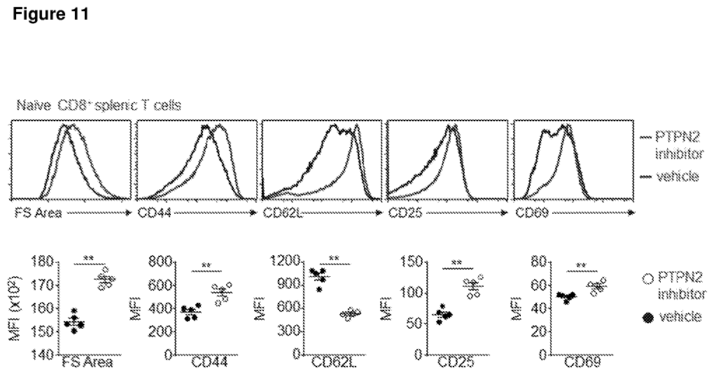

Purified CD8⁺ naive (CD62L^{hi}CD44^{lo}) splenic T cells isolated from C57BL/6 mice where incubated with plate-bound αCD3/CD28 for 48h in the presence or absence (vehicle) of a highly selective reversible PTPN2 inhibitor (compound 8 as describe herein). T cells were stained with fluorochrome-conjugated antibodies to assess CD44, CD62L, IL-2Ra (CD25 subunit) and CD69 surface levels and mean fluorescence intensity (MFI) was monitored by flow cytometry. The results in FIG. 11 show that non-genetic methods of inhibition of PTPN2 can enhance the activity of T cells. The studies described here show that inhibition of PTPN2-activity by a small molecule enhances polyclonal CD8⁺ T cell activation ex vivo.

The results described herein show that the activity of CAR T cells can be enhanced by inhibition of PTPN2. Further, inhibition of PTPN2 in a cell culture of CD8⁺ T cells by a chemical compound enhances polyclonal CD8⁺ T cell activation ex vivo. These results show that inhibition of PTPN2 permits the T helper-independent acquisition of cytotoxic activity which may find particular application for autologous T cell/CAR T cell adoptive cell therapies.

### Example 8

### Mice

Ptpn2^{fl/fl} (C57BL6) and Lck-Cre;Ptpn2^{fl/fl} (C57BL6) or corresponding OT-1 mice or CD45.1 and CD45.1/2 congenic mice were maintained on a 12 h light-dark cycle in a temperature-controlled high barrier facility with free access to food and water. Age- and sex-matched 6-8 week old female recipient mice and 6 week old female donor mice were used for adoptive transfer experiments. For ex-vivo experiments either male or female mice were used. For the generation of Ptpn2^{fl/fl} and Lck-Cre;Ptpn2^{fl/fl} and the corresponding OT-I TCR transgenic mice, Ptpn2^{fl/fl} and Lck-Cre;Ptpn2^{fl/fl} mice or OT-1;Ptpn2^{fl/fl} and OT-1;Lck-Cre;Ptpn2^{fl/fl} mice were mated. For the generation of CD45.1/2 mice, C57BL/6 and CD45.1 mice were mated. The Lck-Cre (originating from James D. Marth's laboratory, UCSD, La Jolla, Calif., USA) and TCR (OT-I and OT-II) transgenic mice on C57BL/6J backgrounds were gifts from W. Alexander (Walter and Eliza Hall Institute) and W. Heath (Walter and Eliza Hall Institute). CD45.1 mice were purchased from the WEHI Animal Facility (Kew, Australia). C57BL/6 human Her-2 transgenic mice were bred at the Peter MacCallum and used for experimentation at 6 to 16 weeks.

### Materials

SIINFEKL and SIYNFEKL peptides were purchased from JPT Peptide Technologies. Hamster α-mouse CD3ε (145-2C11), α-mouse CD28E (37.51) and the FITC-Annexin V Apoptosis Detection Kit I were purchased from BD Biosciences. Fetal bovine serum (HyClone) was purchased from Thermo Scientific and Dulbecco-Phosphate Buffered Saline (D-PBS) and 1640 RPMI from Invitrogen.

### Flow Cytometry

Single cell suspensions from dissected spleens and lymph nodes were obtained by gently compressing between frosted glass slides and washed with cold PBS supplemented with 2% (v/v) fetal bovine serum (FBS; CSL). Cell suspensions were recovered by centrifugation (300×g, 5 min at 4° C.) and cell counts determined using a Z1 Coulter Counter (Beckman Coulter). Hepatic and lung lymphocytes were isolated from perfused livers and lungs cut it into small pieces and strained through a 200-micron sieve followed by a 33% Percoll (GE Healthcare Bio-Sciences) gradient at room temperature. Red blood cells were removed using red blood cell lysing buffer (Sigma-Aldrich).

For surface staining, cells (1×10⁶/10 μl) were resuspended in D-PBS/2% FBS and stained in 96-well microtiter plates (Falcon, BD Biosciences) for 20 minutes on ice. For sorting, cells were stained in 15 ml Falcon tubes (BD Biosciences) for 30 minutes on ice. Cells were washed and resuspended in D-PBS/2% FBS and analysed using a LSRII (BD Biosciences) or CyAn ADP (Beckmann-Coulter) or purified using an Influx sorter (BD Biosciences). Purified CD8⁺CD62L^{hi}CD44^{lo}T cells were routinely tested for purity (>99%). Data was analysed using FlowJo7 (Tree Star Inc.) software.

### Antibodies

The following antibodies from BD Pharmingen (San Jose, Calif.) were used for staining: Fluorescein-isothiocyanate (FITC)-conjugated or BD Horizon™ V450-conjugated α-CD44 (IM7), phycoerythrin (PE)-conjugated or allophycocyanin (APC)-conjugated α-CD62L (MEL-14), Pacific Blue-conjugated or Alexa Fluor 647-conjugated α-CD8 (53-6.7), PE-conjugated α-CD25 (PC61) and PE-cyanine dye 7 (Cy7) α-CD69 (H1.2F3), APC-conjugated human α-CD69 (FN50) and APC-Cy7-conjugated human a-CD154 (TRAP-1). The following antibodies from Miltenyi Biotec (Bergisch-Gladbach, Germany) were used for staining: VioBright FITC-conjugated human α-CD8 (BW135/80) and VioBlue-conjugated human α-CD8 (T6D11).

### RNA Interference in Primary Murine T Cells

Ptpn2 was knocked down transiently in primary murine T cells using Ptpn2 (AAGCCCAUAUGAUCACAGUCG, SEQ ID NO: 14); Dharmacon Thermo Scientific, Waltham, Mass.); green fluorescent protein (GFP; CAAGCUGACCCUGAAGUUCdTdT; Dharmacon Thermo Scientific, Waltham, Mass.) siRNA was used as a control. Primary murine T cells were transfected with 30 nM, 100 nM and 300 nM siRNA using the Amaxa Mouse T cell Nucleofactor Kit (Lonza, Basel, Sitzerland) according to the manufacturer's instructions. To monitor transfection efficiency by flow cytometry splenocytes were also transfected with 30 nM BLOCK-iT™ Fluorescent Oligo (Life Technologies, Carlsbad, Calif.). After overnight incubation Amaxa transfection medium was replaced with 1640 RPMI complete medium and T cells were stimulated in 96-well round-bottom plates with plate-bound α-CD3 (5 μg/ml) and α-CD28 (5 μg/ml) for 48h. T cell numbers were monitored by flow cytometry. For quantification Calibrite Beads (BD Biosciences) were added to the wells before cells were harvested.

### In Vitro Stimulation

CD8⁺CD62L′CD44^{lo} naïve LN T cells (1×10⁵/well) were stimulated in 96-well round-bottom plates with plate-bound α-CD3 (5 μg/ml) and α-CD28 (5 μg/ml) for 60h. PTPN2 inhibitor was added twice daily to the culture. To determine the activation status cells were harvested, stained with fluorochrome-conjugated antibodies against α-CD25, α-CD44, α-CD62L and α-CD69 and analysed by flow cytometry.

### In Vitro Apoptosis

CD8⁺CD62L^{hi}CD44^{lo} naïve LN T cells (2×10⁵/well) from TCR-transgenic OT-1 mice were stimulated in 96-well round-bottom plates with 1 μg/ml SIINFEKL or 1 μg/ml SIYNFEKL for 18h. Cells were harvested and stained with the FITC-Annexin V Apoptosis Detection Kit (BD Biosciences) and propidium iodide (PI)−Annexin V+(apoptotic) cells analysed by flow cytometry. For quantification Calibrite Beads™ (BD Biosciences) were added to the wells before cells were harvested.

### Adoptive Cell Therapy Experiments

C57BL/6 human Her-2 transgenic mice were injected subcutaneously with 1×10⁶ 24JK-Her-2 cells (provided by Dr. Patrick Hwu; NIH, Bethesda, Md.) into the fourth mammary fat pad. Mice were then preconditioned with a sublethal dose of g-irradiation (5 Gy) on day 7 post-tumour injection, before transfer of scFv-anti-Her-2-transduced T cells (1×10⁷). The control group was left untreated. Mice were given twice daily intraperitoneal injections of recombinant human IL-2 (Biological Resource Branch, National Cancer Institute, Frederick, Md.) involving 9 doses of 50,000 IU/200 ml given subsequent T cell transfer.

### Isolation and Stimulation of Human PBMCs

Human blood was collected in 15 ml glass tubes containing Ficoll® gradient (GE Healthcare Life Sciences, Amersham, UK). Peripheral blood monocytes (PBMCs) were recovered by centrifugation (2200×rpm, 20 min at room temperature). PBMCs were incubated with PTPN2 inhibitor (compound 8) in serum-free RPMI 1640 for two hours at 37° C. and stimulated with plate-bound human α-CD3 (5 μg/ml; clone OKT-3) for 24h. Cells were harvested and stained with fluorochrome-conjugated antibodies against α-CD8, α-CD45RA, α-CD154 and α-CD69 and analysed by flow cytometry to determine their activation status. To monitor T cells proliferation Calibrite Beads™ were added to the wells before cells were harvested.

### Results

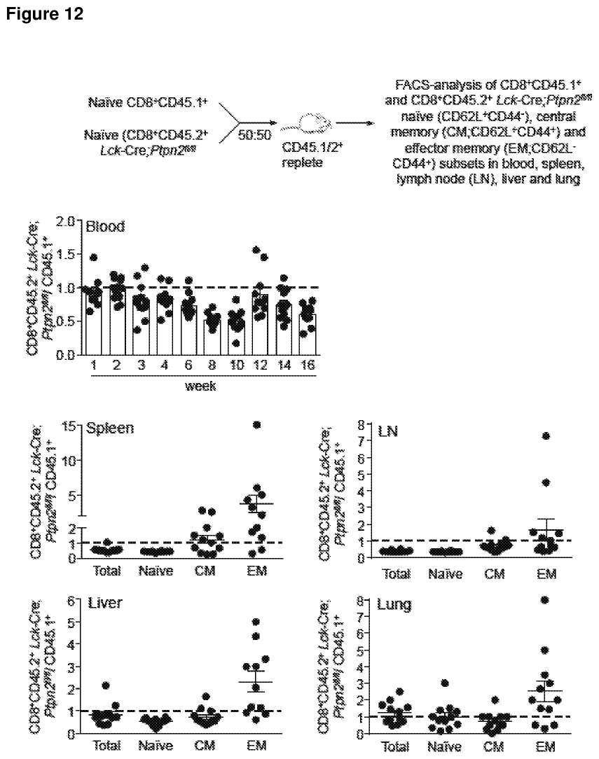

FIG. 12 shows the results from naive (CD62L^{hi}CD44^{lo}) CD8+ LN T cells (2×10⁶) from CD45.1⁺ versus CD45.2⁺ Lck-Cre;Ptpn2^{fl/fl} mice that were co-transferred into replete CD45.1/2⁺ hosts. Peripheral blood was collected at the indicated time points post T cell transfer and the ratios of adoptively transferred CD8⁺ T cells from CD45.2 Lck-Cre;Ptpn2^{fl/fl} versus CD45.1 mice were determined by flow cytometry. At 16 weeks post-transfer recipient mice were sacrificed and lymphocytes from spleen, lymph node (LN), liver and lung were analyzed by flow cytometry. The ratios of adoptively transferred total and naive (CD62L^{hi}CD44^{lo}) central memory (CD62L^{hi}CD44^{hi}; CM) and effector/memory (CD62L^{lo}CD44^{hi}; EM) CD8+ T cells were determined. Results in FIG. 12 shown are means±SEM for the indicated number of mice. These results show enhanced conversion in vivo of naive CD8+ T cells to effector memory type T cells in PTPN2 knockout cells compared to wildtype. It is suspected that the absence of PTPN2 also leads to increased survival of the effector memory phenotype T cells.

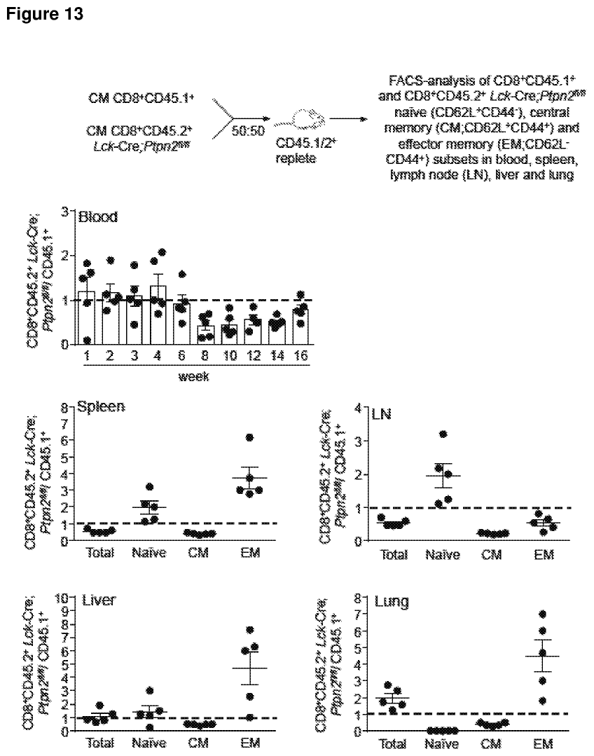

FIG. 13 shows the results of central memory (CD62L^{hi}CD44^{hi}, CM) CD8+ LN T cells (0.5×10⁶) from CD45.1⁺ versus CD45.2⁺ Lck-Cre;Ptpn2^{fl/fl} mice that were co-transferred into replete CD45.1/2+ hosts. Peripheral blood was collected at the indicated time points post T cell transfer and the ratios of adoptively transferred CD8⁺ T cells from CD45.2 Lck-Cre;Ptpn2^{fl/fl} versus CD45.1 mice were determined by flow cytometry. At 16 weeks post-transfer recipient mice were sacrificed and lymphocytes from spleen, lymph node (LN), liver and lung were analyzed by flow cytometry. The ratios of adoptively transferred total and naive (CD62L^{hi}CD44^{lo}) central memory (CD62L^{hi}CD44^{h}; CM) and effector/memory (CD62L^{lo}CD44^{hi}; EM) CD8+ T cells were determined. Results shown are means±SEM for the indicated number of mice. PTPN2-deficiency enhances conversion in vivo of central memory T cells to effector memory phenotype.

Taken together the results in FIGS. 12 and 13 show that a whether the starting population of cells are naive CD8+ T cells, central memory phenotype cells or a mixture of both, transfer into a host allows conversion into effector memory phenotype and that this conversion is enhanced in the absence of PTPN2. Effector memory phenotype cells are a population of cells useful for adoptive transfer, in particular for treating tumours.

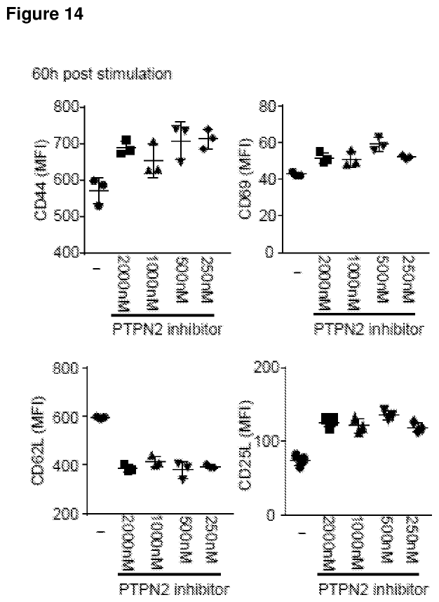

In FIG. 14, the results of naive (CD62L^{hi}CD44^{lo}) CD8+LN T cells (1×10⁵) from C57BL/6 mice that were stimulated with plate-bound anti-CD3 (5 μg/ml) and anti-CD28 (5 μg/ml) for 60h±PTPN2 inhibitor (compound 8) at various concentrations is shown. Cells were harvested and stained with fluorochrome-conjugated antibodies for CD44, CD69, CD25 and CD62L and mean fluorescence intensity (MFI) analyzed by flow cytometry. Results shown are means±SD for the indicated number of replicates. The results in FIG. 14 show that PTPN2 inhibition stimulates naive CD8+ T cells to an activated phenotype.

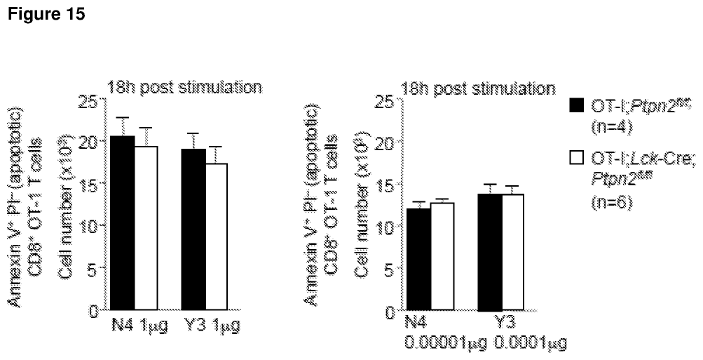

CD8+ naive LN T cells (2×10⁵) from OT-I:Ptpn2^{fl/fl} versus OT-I; Lck-Cre;Ptpn2^{fl/fl} mice were incubated with 1 μg/ml SIINFEKL (N4; high affinity peptide) or 1 μg/ml SIYNFEKL (Y3; low affinity peptide) for 18 h, stained for Annexin V and propidium-iodide (PI) and analysed by flow cytometry (FIG. 15). Results shown are means±SEM for the indicated number of mice. The results in FIG. 15 show that PTPN2 deficiency does not lead to activation-induced cell death. In conjunction with the results shown elsewhere herein, in particular FIGS. 12 to 14, PTPN2 deficiency or inhibition enhances the in vivo conversion of naive or central memory type T cells to an effector memory phenotype without increasing cell death via activation-induced cell death (AICD). AICD is a negative regulator of activated T lymphocytes that results from repeated stimulation of their T-cell receptors (TCR) and helps to maintain peripheral immune tolerance. The experiments shown herein demonstrate that the PTPN2 deficient or inhibited T cells are robust and are not any more susceptible to AICD than wildtype cells.

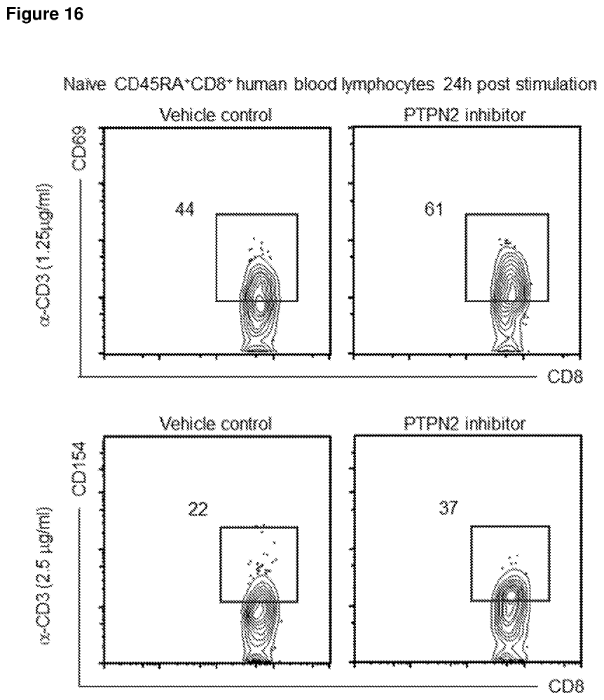

The results in FIG. 16 show that the inhibition of PTPN2 by a small molecule inhibitor, compound 8, in CD8+ human blood lymphocytes results in enhanced TCR-mediated T cell activation. Freshly isolated human PBMCs (2×10⁶) were stimulated with plate-bound α-CD3 (1.25 and 2.5 μg/ml) for 24h. PBMCs were harvested and stained with fluorochrome-conjugated antibodies for CD45RA, CD8, CD69 and CD154 and T cell activation was monitored by flow cytometry. Representative CD8 versus CD69 and CD154 plots (numbers in outlined areas are the relative numbers in the gate) are shown (FIG. 16). These data confirm that inhibition of PTPN2 in human CD8+ cells, including cells isolated from peripheral blood, exhibit enhanced T cell receptor mediated T cell activation.

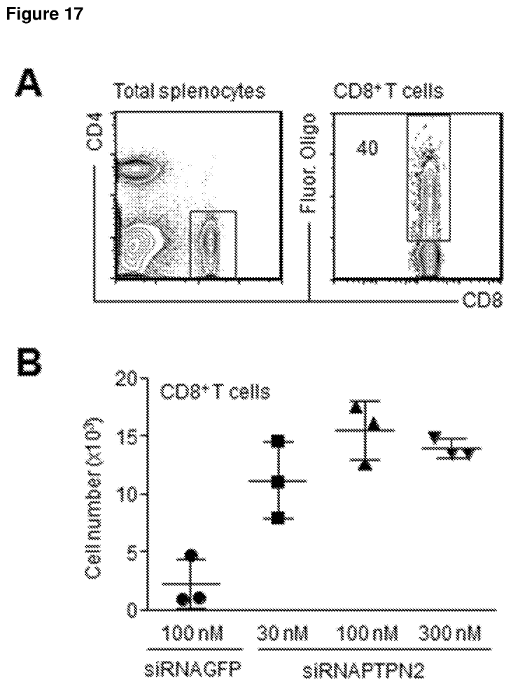

Knock down of PTPN2 using siRNAs in murine CD8+ T cells lead to enhanced TCR-mediated T cell responses, shown by a dose response increase in cell number (FIG. 17). Splenocytes (1×10⁷) from C57BL/6 mice were transfected overnight with 100 nM GFP siRNA or 30 nM, 100 nM and 300 nM PTPN2 siRNA using the Amaxa Mouse T cell Nucleofactor Kit. Transfected splenocytes were stimulated with plate-bound α-CD3 (5 μg/ml) and α-CD28 (5 μg/ml) for 48h and stained with fluorochrome-conjugated α-CD8. CD8+ T cell numbers were quantified by flow cytometry. Results shown are means±SD for the indicated number of replicates.

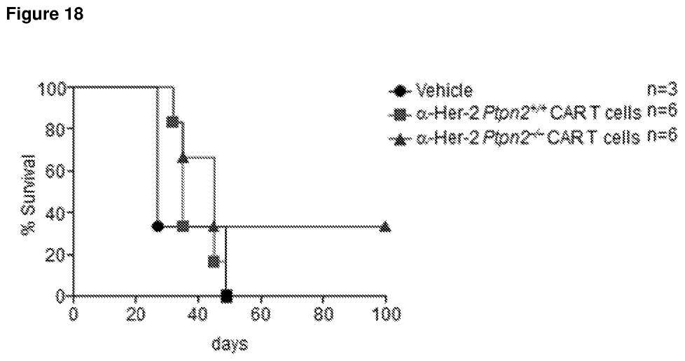

PTPN2-deficiency enhances the tumour-specific activity of Her-2 specific CAR T cells in the context of adoptive immunotherapy and prolongs the survival of xenografted mice (FIG. 18). Her-2-specific Ptpn2^{fl/fl} (α-Her-2+/+) versus Lck-Cre; Ptpn2fl/fl CAR T cells (α-Her-2−/−) (1×10⁷) were adoptively transferred into C57BL/6 human Her-2 transgenic mice (6 mice in each group) infected with Her-2 expressing 24JK-Her-2 sarcoma cells and survival was monitored. After 100 days, two out of six mice that received the adoptive transfer of Lck-Cre; Ptpn2^{fl/fl} CAR T cells (α-Her-2−/−) were still alive.

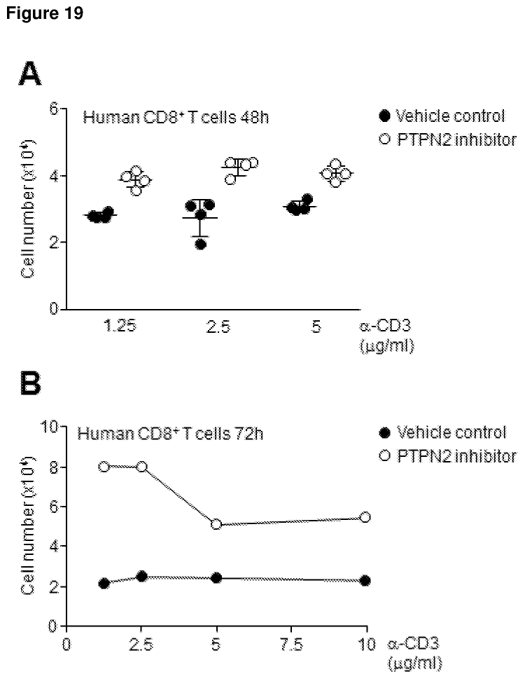

Inhibition of PTPN2 in CD8⁺ human blood lymphocytes results in enhanced TCR-mediated T cell proliferation (FIG. 19). Freshly isolated human PBMCs (2×10⁶) were stimulated with plate-bound α-CD3 for (A) 48h and (B) 72h, in the presence of vehicle control or the PTPN2 inhibitor, compound 8 (as described herein). PBMCs were harvested and stained with fluorochrome-conjugated α-CD8. Calibrite Beads™ were added and T cell proliferation was monitored by flow cytometry. Results shown are means±SD for the indicated number of replicates.

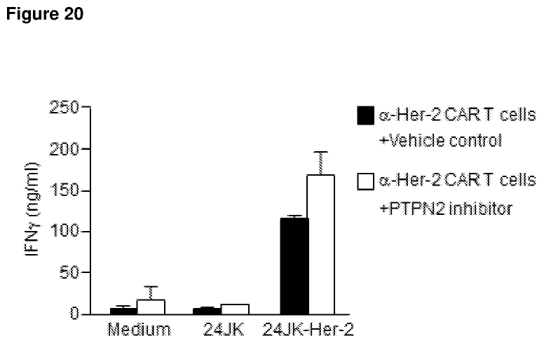

Inhibition of PTPN2 enhances the tumour-specific activation of Her-2 specific CAR T cells ex vivo (FIG. 20). Her-2-specific CAR T cells were incubated with Her-2 expressing 24JK sarcoma cells (24JK-Her-2) or 24JK sarcoma cells (24JK) or medium alone, in the presence of vehicle control or the PTPN2 inhibitor, compound 8 (described herein). T cell activation was assessed by monitoring for secreted IFNγ by ELISA (Mouse IFNγELISA Set, BD OptEIA™) according to the supplier's specifications. Tests were performed in triplicates (±SD).

1.-37. (canceled)

38. A method for producing a CD8+ CAR T cell that has an enhanced capacity for direct killing of a cancer cell, wherein the CD8+ CAR T has specificity for an antigen expressed by a cancer cell, the method including:
providing a CD8+ CAR T has specificity for an antigen expressed by a cancer cell;
subjecting the CD8+ CAR T cell to genome editing to remove or modify all or part of a sequence encoding PTPN2 in the CD8+ CAR T cell,
thereby producing a CD8+ CAR T cell that has an enhanced capacity for direct killing of a cancer cell compared to a CD8+ CAR T cell which has not been subjected to genome editing to remove or modify all or part of a sequence encoding PTPN2.

39. The method according to claim 38, wherein the CD8+ CAR T cell is derived from a subject having a cancer.

40. The method according to claim 38 wherein the only genome editing in the CD8+ CAR T cell occurs in the gene encoding PTPN2.

41. The method according to claim 38 wherein the CD8+ CAR T cell has specificity for the antigen Her-2.

42. An isolated, purified or recombinant CD8+ CAR T cell produced by the method according to claim 38.

43. A composition comprising a cell according to claim 43 and a pharmaceutically acceptable carrier.

44. The composition according to claim 43, further including a cytokine for enhancing cell killing.

45. A method for treating cancer including the steps of:
culturing CD8+ CAR T cells specific for a tumour antigen expressed by the cancer to be treated;
subjecting the CD8+ CAR T cells to genome editing to remove or modify all or part of a sequence encoding PTPN2 in the CD8+ CAR T cells,
administering the genome edited CD8+ CAR T cells to a subject in need thereof,
thereby treating cancer in the subject.

46. The method according to claim 45, wherein treating the cancer is selected from the group consisting of
reducing the number of cancer cells;
reducing the primary tumour size;
inhibiting cancer cell infiltration into peripheral organs;
inhibiting tumour metastasis;
inhibiting tumour growth;
promoting regression of the cancer;
prolonging survival of the subject; and
relieving one or more of the symptoms associated with the cancer.

47. The method according to claim 45, wherein the only genome editing in the CD8+ CAR T cell occurs in the gene encoding PTPN2.

48. The method according to claim 48, wherein the CD8+ CAR T cell is derived from a subject having cancer.

49. The method according to claim 45, wherein the CD8+ CAR T cell is derived from the subject requiring treatment for cancer.

50. The method according to claim 45, wherein the CD8+ CAR T cell is isolated from an allogenic donor.

51. The method according to claim 45, wherein the cancer is a Her-2 positive cancer and the CD8+ CAR T cell is specific for Her-2.

52. The method according to claim 38, wherein the CD8+ CAR T cell is expanded in culture before or after being subjected to the genome editing.

53. The method according to claim 46, wherein the CD8+ CAR T cell is administered in the absence of a T helper cell.

54. The method according to claim 46, wherein the CD8+ CAR T cell is administered in the absence of a cytokine.
